SEQUENCE LISTING (14 sequences)

SEQ ID NO: 1 - DNA, 1764 bp, Artificial Sequence
  ACE2t DNA sequence (codon optimized)
cagagcacaattgaggaacaggccaagaccttcctggacaagttcaaccacgaggccgaggacctgttctaccagtctagcctggccagctggaactacaacaccaacatcaccgaagagaacgtgcagaacatgaacaacgccggcgacaagtggagcgccttcctgaaagagcagagcacactggcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcctgggatgcccagcggatcttcaaagaggccgagaagttcttcgtgtccgtgggcctgcctaatatgacccaaggcttctgggagaactccatgctgacagaccccggcaatgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccacgaagccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgcccttcacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaatatgaacgtgcggccactgctgaactacttcgagcccctgttcacctggctgaaggaccagaacaagaacagctttgtcggc

SEQ ID NO: 2 - protein, 588 aa, Artificial Sequence
  ACE2t protein sequence
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 3 - DNA, 684 bp, Artificial Sequence
  Modified Fc DNA sequence
tctgataagacccacacctgtcctccatgtcctgctccagaactgctcggcggaccttccgtgttcctgtttcctccaaagcctaaggacaccctgatgatcagcagaacccctgaagtgacctgcgtggtggtggccgtgtctcacgaagatcccgaagtgaagttcaattggtacgtcgacggcgtggaagtgcacaacgccaagaccaagcctagagaggaacagtacgccagcacctacagagtggtgtccgtgctgactgtgctgcaccaggattggctgaacggcaaagagtacaagtgcaaggtgtccaacaaggccctgccagctcctatcgagaaaaccatcagcaaggccaagggccagcctagggaaccccaggtttacacactgcctccaagcagggacgagctgaccaagaatcaggtgtccctgacctgcctggtcaagggcttttacccctccgacattgccgtggaatgggagagcaatggccagcctgagaacaactacaagaccacacctcctgtgctggacagcgacggctcattcttcctgtactccaagctgaccgtggacaagtccagatggcagcagggcaacgtgttcagctgcagcgtgatgcacgaggccctgcacaatcactacacccagaagtccctgtctctgagccctggcaaa

SEQ ID NO: 4 - protein, 228 aa, Artificial Sequence
  Modified Fc amino acid sequence
SDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 33 bp, Artificial Sequence
  gp120t nucleotide sequence
tcctcaggaggggacccagaaattgtaacgcac

SEQ ID NO: 6 - protein, 11 aa, Artificial Sequence
  gp120t amino acid sequence
SSGGDPEIVTH

SEQ ID NO: 7 - protein, 4 aa, Artificial Sequence
  linker sequence
GGGS

SEQ ID NO: 8 - protein, 7 aa, Artificial Sequence
  linker sequence
SSGGGGS

SEQ ID NO: 9 - protein, 15 aa, Artificial Sequence
  linker sequence
SSGGGGSGGGGGGSS

SEQ ID NO: 10 - protein, 18 aa, Artificial Sequence
  linker sequence
SSGGGGSGGGGGGSSRSS

SEQ ID NO: 11 - protein, 32 aa, Artificial Sequence
  CD8a transmembrane domain
FACDIYIWAPLAGTCGVLLLSLVITLYCNHRN

SEQ ID NO: 12 - protein, 43 aa, Artificial Sequence
  CD8a hinge region
KPTTTPAPRPPTPAPTIASQPLSLRPEACRPAAGGAVHTRGLD

SEQ ID NO: 13 - protein, 230 aa, Artificial Sequence
  hinge-CH2-CH3 region of antibody isotype IgG4
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 14 - protein, 112 aa, Artificial Sequence
  CD3zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0242643 A1
(43) Pub. Date: Aug. 3, 2023

(54) ADAPTER MOLECULES TO RE-DIRECT CAR T CELLS TO AN ANTIGEN OF INTEREST

(71) Applicants: ANTION BIOSCIENCES SA, Geneva (CH); GENEVA UNIVERSITY HOSPITALS, Geneva (CH); UNIVERSITY OF GENEVA, Geneva (CH); UNIVERSITY OF ZURICH, Zurich (CH)

(72) Inventors: Marco ALESSANDRINI, Geneva (CH); Karl-Heinz KRAUSE, Geneva (CH); Renier MYBURGH, Zurich (CH)

(73) Assignees: ANTION BIOSCIENCES SA, Geneva (CH); GENEVA UNIVERSITY HOSPITALS, Geneva (CH); UNIVERSITY OF GENEVA, Geneva (CH); UNIVERSITY OF ZURICH, Zurich (CH)

(21) Appl. No.: 17/999,735

(22) PCT Filed: May 27, 2021
(86) PCT No.: PCT/IB2021/000358

§ 371 Date: Nov. 23, 2022

Related U.S. Application Data
(60) Provisional application No. 63/030,653, filed on May 27, 2020.

(51) Int. Cl. C07K 16/28

(52) U.S. Cl. CPC C07K 16/2809; A61K 2039/505

(57) ABSTRACT

Chimeric antigen receptor (CAR) bridging protein are provided comprising a CAR-binding domain linked to an antigen-binding domain, which can be used to re-direct the targeting of CAR-T cells. The bridging protein may comprie an antigen-binding domain that targets any antigen of interest, such as, for example, a tumor antigen or viral antigen. Also provided are methods of using the bridging proteins in combination with CAR-T cells to treat a disease, such as, for example, a cancer or an infectious disease.

### REFERENCE TO RELATED APPLICATIONS

The present application claims the priority benefit of U.S. Provisional App. No. 63/030,653, filed May 27, 2020, the entire contents of which is incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on May 26, 2021, is named ANBOP0005WO_ST25.txt and is 16.4 kilobytes in size.

### BACKGROUND

### 1. Field

The present disclosure relates generally to the fields of immunology, virology, and medicine. More particularly, it concerns bridging proteins that re-direct CAR-expressing immune effector cells to any antigen of interest, and methods of using the same to treat disease.

### 2. Description of Related Art

Recently engineered immune effector cells have become an attractive therapeutic for the treatment of viral disease and cancer. For example, T cells can be engineered to express chimeric antigen receptors (CARs) that target any particular antigen of interest. Such cells enable targeted killing of cell that express cancer markers or any infected with pathogens. Despite the promise of these new therapies, issue remain with off target toxicities and lack of persistence of engineered cells in treated subjects. For example, tumor heterogeneity and the loss of target antigen expression is a significant challenge for developing effective chimeric antigen receptor (CAR) T-cell therapies. Very few tumor-specific antigens exist (i.e., those expressed exclusively on tumor cells), while most are tumor-associated (i.e., over-expressed on tumor cells, but to a lesser extent on healthy cells). Tumors also have the propensity to lose expression of CAR-targeted antigens, and thus many groups are developing bi-specific and tri-specific CAR T cells in order to capture a greater diversity of tumor cells. This is well-described in the context of B-cell malignancies, where multi-specific CAR T cells against CD19, CD20 and CD22 are in clinical development. Solid tumors and the solid tumor microenvironment are an even greater challenge to overcome with considerably more tumor heterogeneity. New, more advanced methods, of targeting immune effector cells are thus in great need.

### SUMMARY

Provided herein are chimeric antigen receptor (CAR) bridging proteins that redirect mono-specific CAR-T cells to alternative or multiple target antigens. For example, fusion proteins and antibody-conjugates are provided, which, on the one end, engage a CAR and, on the other, a target antigen of choice. Therefore, in contrast to creating multi-specific CARs, the bridging protein provided herein re-direct single-variant CAR-T cells toward diverse antigens via multi-specific bridge proteins. Single or multiple bridge proteins can be infused either sequentially or together as a moiety for a simultaneous multi-targeted approach.

In some embodiments, the present disclosure provides chimeric antigen receptor (CAR) bridging proteins comprising (1) an antigen-binding domain and (2) a CAR-binding domain, that comprises at least a portion of an HIV-1 gp120 protein. In some aspects, the CAR-binding domain is chemically conjugated to the antigen-binding domain. In some aspects, the CAR bridging proteins further comprise an antibody Fc domain. In some aspects, the Fc domain is positioned between the CAR-binding domain and the antigen-binding domain. In some aspects, the CAR-binding domain is positioned between the antigen-binding domain and the Fc domain. In some aspects, the CAR bridging proteins further comprise a linker sequence between the antigen binding domain and the CAR-binding domain. In some aspects, the CAR-binding domain comprises the sequence provided in SEQ ID NO: 6. In some aspects, the Fc domain comprises a human Fc domain sequence. In other aspects, the Fc domain comprises a human heavy chain Fc domain sequence. In still other aspects, the Fc domain comprises CH2 and CH3 regions of a human heavy chain Fc domain sequence. In yet other aspects, the Fc domain comprises substitutions relative to the wild-type human heavy chain Fc domain sequence which prevent binding to FcgR receptors. In other aspects, the Fc domain comprises a sequence that is at least 85%, at least 90%, at least 95%, or 100% identical to the sequence provided by SEQ ID NO: 4.

In some aspects, the antigen-binding domain binds to a tumor antigen or a viral antigen. In some aspects, the antigen-binding domain comprises a peptide that interacts with an antigen of interest. In some aspects, the antigen-binding domain comprises an antigen-binding portion of an antibody that recognizes the antigen of interest. In some aspects, the antigen-binding domain comprises at least a portion of a ligand that interacts with the antigen of interest. In some aspects, the antigen-binding domain is capable of binding to CD19, CD20, or CD22. In other aspects, the antigen-binding domain is capable of binding to a coronavirus spike protein. In further aspects, the coronavirus spike protein is a SARS-CoV-1 or SARS-CoV-2 spike protein. In some aspects, the antigen-binding domain comprises at least a portion of an ACE2 extracellular domain. In further aspects, the portion of an ACE2 extracellular domain is the ACE2t domain. In still further aspects, the ACE2t domain comprises a sequence that is at least 85%, at least 90%, at least 95%, or 100% identical to the sequence of SEQ ID NO: 2.

In some aspects, the CAR bridging proteins further comprise at least one linker sequence between the CAR-binding domain, Fc domain, and/or antigen-binding domain. In some aspects, the CAR bridging protein comprises a linker sequence between each of the CAR-binding domain, Fc domain, and/or antigen-binding domains. In some aspects, the linker sequence comprises the sequence of GGGS (SEQ ID NO: 7). In some aspects, the linker sequence comprises a sequence provided by SEQ ID NO: 8. In some aspects, the CAR bridging protein forms a homodimer.

In other embodiments, the present disclosure provides chimeric antigen receptor (CAR) bridging proteins comprising a CAR-binding domain and an antigen-binding domain. In some aspects, the CAR-binding domain is chemically conjugated to the antigen-binding domain. In some aspects, the CAR bridging proteins further comprising an antibody Fc domain. In some aspects, the Fc domain is positioned between the CAR-binding domain and the antigen-binding domain. In other aspects, the CAR-binding domain is positioned between the antigen-binding domain and the Fc domain. In some aspects, the CAR-binding domain comprises a peptide that interacts with the extracellular portion of a CAR. In some aspects, the CAR-binding domain comprises the antigen-binding portion of an antibody that recognizes the extracellular portion of a CAR. In some aspects, the CAR-binding domain comprises at least a portion of a ligand that interacts with the extracellular portion of a CAR. In some aspects, the CAR-binding domain comprises at least a portion of an HIV-1 gp120 protein. In further aspects, the CAR-binding domain comprises the sequence provided in SEQ ID NO: 6. In certain aspects, the CAR-binding domain consists essentially of the sequence provided in SEQ ID NO: 6. In certain aspects, the CAR-binding domain consists of the sequence provided in SEQ ID NO: 6.

In some aspects, the Fc domain comprises a human Fc domain sequence. In some aspects, the Fc domain comprises a human heavy chain Fc domain sequence. In some aspects, the Fc domain comprises CH2 and CH3 regions of a human heavy chain Fc domain sequence. In some aspects, the Fc domain comprises substitutions relative to the wild-type human heavy chain Fc domain sequence which prevent binding to FcgR receptors. In some aspects, the Fc domain comprises a sequence that is at least 85%, at least 90%, at least 95%, or 100% identical to the sequence provided by SEQ ID NO: 4. In some aspects, the antigen-binding domain binds to a tumor antigen or a viral antigen. In some aspects, the antigen-binding domain comprises a peptide that interacts with an antigen of interest. In some aspects, the antigen-binding domain comprises an antigen-binding portion of an antibody that recognizes the antigen of interest. In some aspects, the antigen-binding domain comprises at least a portion of a ligand that interacts with the antigen of interest. In some aspects, the antigen-binding domain is capable of binding to CD19, CD20, or CD22. In some aspects, the antigen-binding domain is capable of binding to a coronavirus spike protein. In further aspects, the coronavirus spike protein is a SARS-CoV-1 or SARS-CoV-2 spike protein.

In some aspects, the antigen-binding domain comprises at least a portion of an ACE2 extracellular domain. In some aspects, the portion of an ACE2 extracellular domain is the ACE2t domain. In further aspects, the ACE2t domain comprises a sequence that is at least 85%, at least 90%, at least 95%, or 100% identical to the sequence of SEQ ID NO: 2. In some aspects, the CAR bridging proteins further comprise at least one linker sequence between the CAR-binding domain, Fc domain, and/or antigen-binding domain. In some aspects, the CAR bridging protein comprises a linker sequence between the CAR-binding domain and the antigen-binding domain, and optionally, the Fc domain. In some aspects, the linker sequence comprises the sequence of GGGS (SEQ ID NO: 7). In some aspects, the linker sequence comprises a sequence provided by SEQ ID NO: 8. In some aspects, the CAR bridging protein forms a homodimer.

In still other embodiments, the present disclosure provides nucleic acid molecules encoding a CAR bridging protein of the present disclosure. In some aspects, the sequence encoding the CAR bridging protein is operatively linked to an expression control sequence. In some aspects, the nucleic acid molecules are further defined as an expression vector. In some aspects, the expression vector is an episomal vector. In other aspects, the expression vector is a viral vector. In further aspects, the viral vector is an adenovirus, adeno-associated virus, retrovirus or lentivirus vector.

In yet other embodiments, the present disclosure provides pharmaceutical compositions comprising a CAR bridging protein of the present disclosure in a pharmaceutically acceptable carrier. In some aspects, the pharmaceutical compositions further comprise a population of immune effector cells comprising a CAR polypeptide that the CAR-binding domain of the CAR bridging protein binds.

In further embodiments, the present disclosure provides methods of treating a subject in need thereof, the method comprising administering to the subject an effective amount of a CAR bridging protein of the present disclosure. In some aspects, the subject has previously been administered a population of immune effector cells comprising a CAR polypeptide that the CAR-binding domain of the CAR bridging protein binds. In some aspects, the methods further comprise administering to the subject an effective amount of a population of immune effector cells comprising a CAR polypeptide that the CAR-binding domain of the CAR bridging protein binds. In some aspects, the cells are allogeneic to the subject. In some aspects, the cells are autologous to the subject. In some aspects, the cells are HLA matched to the subject. In some aspects, the subject has a coronavirus infection. In some aspects, the subject has a SAR-CoV infection. In some aspects, the subject has a SAR-CoV-2 infection. In some aspects, the subject has COVID-19. In some aspects, the CAR bridging protein comprises (i) an antigen-binding domain that is at least 85%, at least 90%, at least 95%, or 100% identical to the sequence of SEQ ID NO: 2; and (ii) a CAR-binding domain that is comprises the sequence provided in SEQ ID NO: 6, and wherein the CAR polypeptide comprises a CD4 domain as its antigen-binding domain. In some aspects, the subject has a cancer. In some aspects, the CAR bridging protein comprises an antigen-binding domain that is capable of binding to CD19, CD20, or CD22.

In still further embodiments, the present disclosure provides chimeric antigen receptor (CAR) bridging proteins comprising a CAR-binding domain and an antigen-binding domain. In some aspects, the antigen-binding domain is chemically conjugated to the CAR-binding domain. In some aspects, the antigen-binding domain and the CAR-binding domain are comprised in a fusion protein. In some aspects, the CAR bridging protein further comprises an antibody Fc domain. In some aspects, the Fc domain is positioned between the CAR-binding domain and the antigen-binding domain. In other aspects, the CAR-binding domain is positioned between the antigen-binding domain and the Fc domain. In some aspects, the CAR-binding domain comprises a peptide that interacts with the extracellular portion of a CAR. In some aspects, the CAR-binding domain comprises the antigen-binding portion of an antibody that recognizes the extracellular portion of a CAR. In some aspects, the CAR-binding domain comprises at least a portion of a ligand that interacts with the extracellular portion of a CAR. In some aspects, the CAR-binding domain binds to a portion of the CAR that is specific for the target of the CAR. In some aspects, the CAR comprises scFv and wherein the CAR-binding domain binds to a variable region of the scFv. In some aspects, the CAR-binding domain comprises an antibody or an antigen binding fragment thereof. In some aspects, the CAR-binding domain comprises scFv.

In some aspects, the CAR-binding domain comprises at least a portion of an HIV-1 gp120 protein. In some aspects, the CAR-binding domain comprises the sequence provided in SEQ ID NO: 6. In some aspects, the CAR is a CD19 specific CAR and the CAR binding domain binds to the CD19-specific CAR. In some aspects, the CAR binding domain comprises an antibody or an antigen binding fragment thereof. In some aspects, the CAR binding domain comprises a scFv. In some aspects, the CAR-binding domain comprises at least a portion of a CD19 protein. In some aspects, the Fc domain comprises a human Fc domain sequence. In some aspects, the Fc domain comprises a human heavy chain Fc domain sequence. In some aspects, the Fc domain comprises CH2 and CH3 regions of a human heavy chain Fc domain sequence. In some aspects, the Fc domain comprises substitutions relative to the wild-type human heavy chain Fc domain sequence which prevent binding to FcgR receptors. In some aspects, the Fc domain comprises a sequence that is at least 85%, at least 90%, at least 95%, or 100% identical to the sequence provided by SEQ ID NO: 4. In some aspects, the antigen-binding domain binds to a tumor antigen or a viral antigen.

In some aspects, the antigen-binding domain comprises a peptide that interacts with an antigen of interest. In some aspects, the antigen-binding domain comprises an antigen-binding portion of an antibody that recognizes the antigen of interest. In some aspects, the antigen-binding domain comprises at least a portion of a ligand that interacts with the antigen of interest. In some aspects, the antigen-binding domain binds to CD19, CD20, or CD22. In some aspects, the antigen-binding domain is capable of binding to a coronavirus spike protein. In some aspects, the coronavirus spike protein is a SARS-CoV-1 or SARS-CoV-2 spike protein. In some aspects, the antigen-binding domain comprises at least a portion of an ACE2 extracellular domain. In some aspects, the portion of an ACE2 extracellular domain is the ACE2t domain. In some aspects, the ACE2t domain comprises a sequence that is at least 85%, at least 90%, at least 95%, or 100% identical to the sequence of SEQ ID NO: 2. In some aspects, the CAR bridging protein further comprises at least one linker sequence between the CAR-binding domain, Fc domain, and/or antigen-binding domain. In some aspects, the CAR bridging protein comprises a linker sequence between the CAR-binding domain and the antigen-binding domain, and optionally, the Fc domain. In some aspects, the linker sequence comprises the sequence of GGGS (SEQ ID NO: 7). In some aspects, the linker sequence comprises a sequence provided by SEQ ID NO: 8. In some aspects, the CAR bridging protein forms a homodimer.

In yet other embodiments, the present disclosure provides nucleic acid molecule encoding a CAR bridging protein of the present disclosure. In some aspects, the sequence encoding the CAR bridging protein is operatively linked to an expression control sequence. In other aspects, the CAR bridging protein is further defined as an expression vector. In some aspects, the expression vector is an episomal vector. In other aspects, the expression vector is a viral vector. In some aspects, the viral vector is an adenovirus, adeno-associated virus, retrovirus or lentivirus vector.

In other embodiments, the present disclosure provides pharmaceutical compositions comprising a CAR bridging protein of the present disclosure in a pharmaceutically acceptable carrier. In some aspects, the pharmaceutical compositions further comprise a population of immune effector cells comprising a CAR polypeptide that the CAR-binding domain of the CAR bridging protein binds.

In still other embodiments, the present disclosure provides methods of treating a subject in need thereof, the method comprising administering to the subject an effective amount of a CAR bridging protein of the present disclosure. In some aspects, the subject has previously been administered a population of immune effector cells comprising a CAR polypeptide that the CAR-binding domain of the CAR bridging protein binds. In some aspects, the methods further comprise administering to the subject an effective amount of a population of immune effector cells comprising a CAR polypeptide that the CAR-binding domain of the CAR bridging protein binds. In some aspects, the cells are allogeneic to the subject. In some aspects, the cells are autologous to the subject. In some aspects, the cells are HLA matched to the subject. In some aspects, the subject has a coronavirus infection. In some aspects, the subject has a SAR-CoV infection. In other aspects, the subject has a SAR-CoV-2 infection. In still other aspects, the subject has COVID-19. In some aspects, the CAR bridging protein comprises (i) an antigen-binding domain that is at least 85%, at least 90%, at least 95%, or 100% identical to the sequence of SEQ ID NO: 2; and (ii) a CAR-binding domain that is comprises the sequence provided in SEQ ID NO: 6, and wherein the CAR polypeptide comprises a CD4 domain as its antigen-binding domain. In certain aspects, the CAR-binding domain consists essentially of the sequence provided in SEQ ID NO: 6. In certain aspects, the CAR-binding domain consists of the sequence provided in SEQ ID NO: 6. In some aspects, the subject has a cancer. In some aspects, the CAR bridging protein comprises an antigen-binding domain that is capable of binding to CD19, CD20, or CD22. In some aspects, the CAR-binding domain of the CAR bridging protein comprises at least a portion of a CD19 protein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E. Schematic representations of a bridging protein that redirects CAR T cells. FIG. 1A illustrates the general concept of redirecting a CD4 CAR T cell to a target cell using a bridging protein. FIG. 1B illustrates the specificities of the CD4 CAR T cell both acting directly and acting through a bridging protein. FIG. 1C illustrates a bridging protein that redirects CD4 CAR T cells to CoV infected cells. FIG. 1D illustrates the simultaneous or sequential targeting of tumors, which can be used to target a variety of malignant cells or to overcome antigen loss employed by tumor cells to evade targeted therapies. FIG. 1E illustrates the general concept of redirecting a CD19-specific CAR T cell to a target cell using a bridging protein.

FIGS. 2A-2C. Schematic representation of exemplary bridging proteins. FIG. 2A illustrates dimeric bridging proteins having an antigen-binding domain, an Fc region, and a CAR-binding domain. FIG. 2B illustrates a method of conjugating the CAR-binding domain (as represented by gp120t) to an IgG antibody. FIG. 2C illustrates various embodiments of bridging proteins that have a CAR-binding domain (as represented by gp120t), and Fc region, and an antigen-binding domain.

FIG. 3. Schematic representation of the CD4-specific CAR T cell.

FIGS. 4A-4C. Further schematic representations of exemplary bridging proteins. FIG. 4A show a representative method for retargeting CD19-specific CAR cells. FIG. 4B illustrates dimeric bridging proteins having an antigen-binding domain, an Fc region, and a CD-19 CAR-binding domain, such as CD19, truncated CD19 (that binds to the CAR) or an antibody domain specific for a CD19 CAR. FIG. 4C illustrates a method of conjugating the CAR-binding domain (as represented by CD19t) to an IgG antibody.

FIG. 5. Anti-HIV CAR construct showing all the elements of the CAR construct used to produce CAR-T cells targeting HIV env.

FIG. 6. Chemical conjugation of the CD4 binding loop of gp120 to an IgG antibody. The sequence of the gp120 CD4 binding loop (SSGGDPEIVTH) is provided in SEQ ID NO: 6.

FIGS. 7A-7D. Development and testing of bridge protein concept. FIG. 7A illustrates that IgG conjugated with the CD4 binding loop of gp120 (gp120t), as well as FACS contour plots demonstrating the binding of bridge protein to CD4 receptors on primary T-cells. FIG. 7B provides FACS histograms and median fluorescent intensity (MFI) of CAR4-bound bridge protein. FIG. 7C provides a schematic description of experiment where CAR4 T cells were re-directed to tumour cells via both IgG and diabody conjugated antibodies. FIG. 7D illustrates the percentage viable tumour cells following a 24-hour co-culture with CAR4 T cells alone, CAR4 T cells with IgG, Car4 T cells with IgG-gp120t conjugate, and CAR4 T cells with diabody-gp120t conjugate.

### DETAILED DESCRIPTION

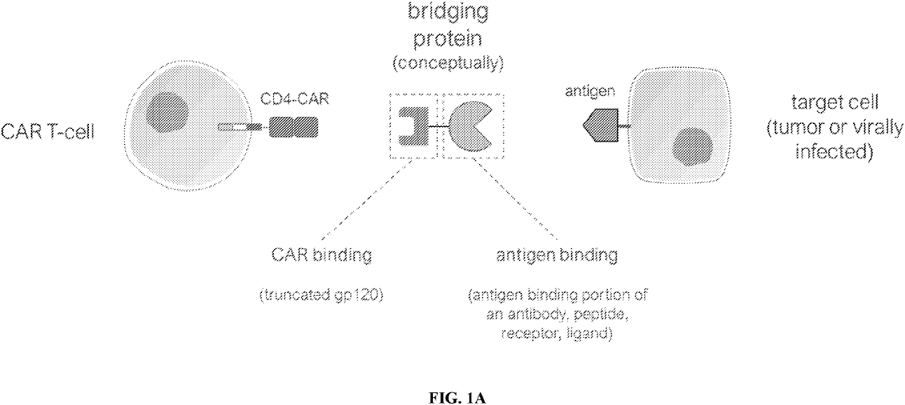

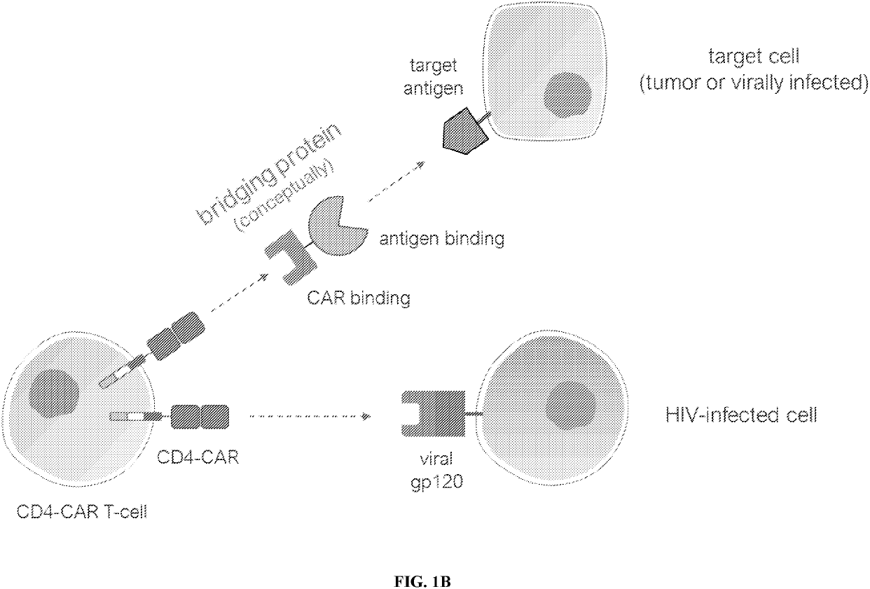

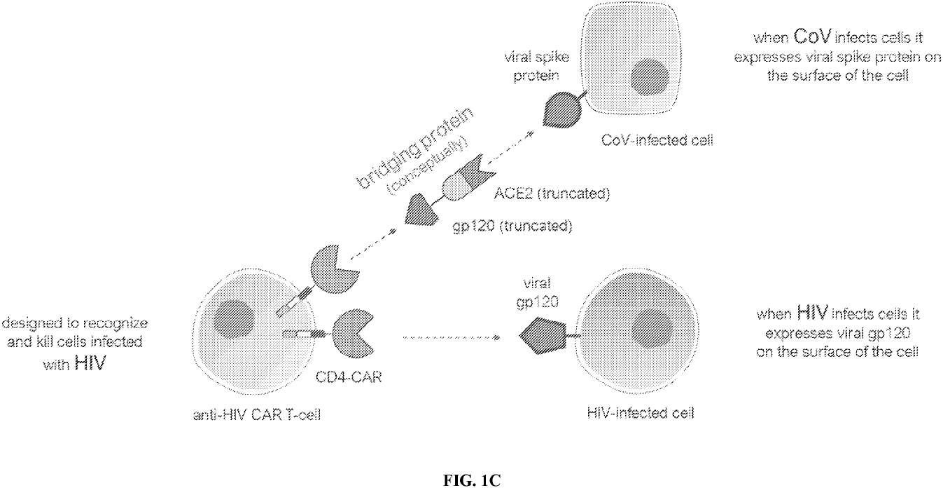

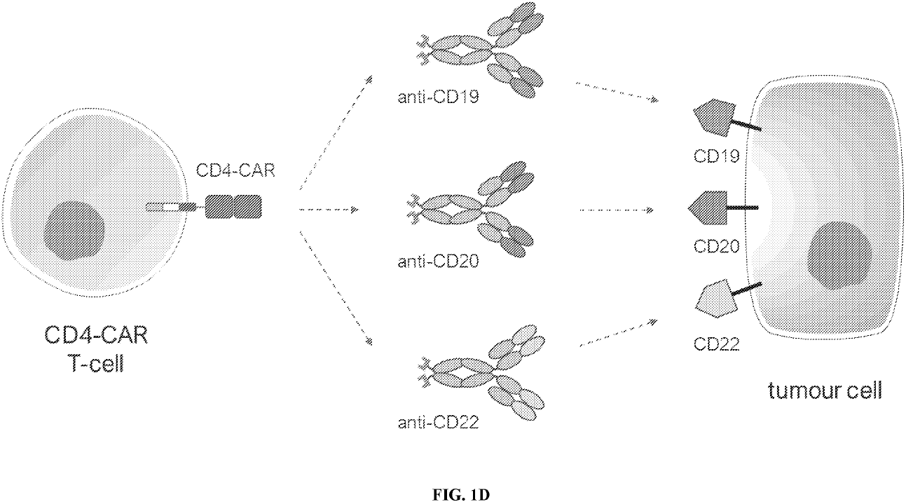

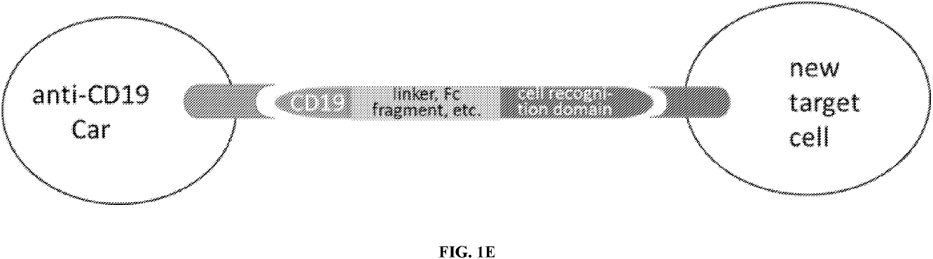

Provided herein are bridging proteins that can be used to redirect CAR-T cells, such as, for example, therapeutic CD4-specific CAR-T cells that are designed to recognize and kill HIV-infected cells. In that example, the bridging proteins may comprise a truncated gp120 extracellular domain fused to a protein domain that binds to the target antigen of interest (FIGS. 1A and 1B). For example, the protein domain may be an ACE2 extracellular domain (the natural receptor used by CoV to infect human cells). When CoV infects a cell, viral spike protein is expressed on the surface of the cell. Thus, in the presence of the gp120-ACE2 bridging protein, the CD4-specific CAR-T cell will bind to viral spike protein present on the surface of CoV-infected cells (FIG. 1C).

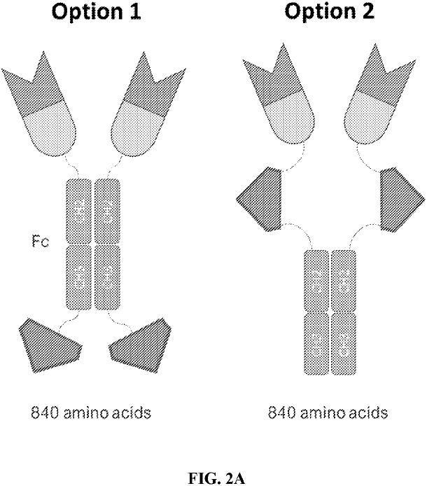

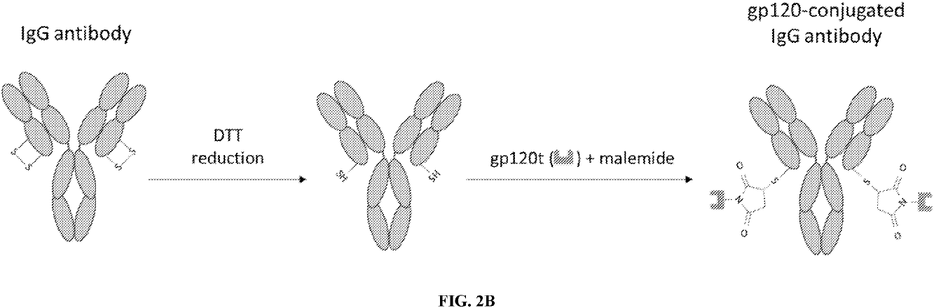

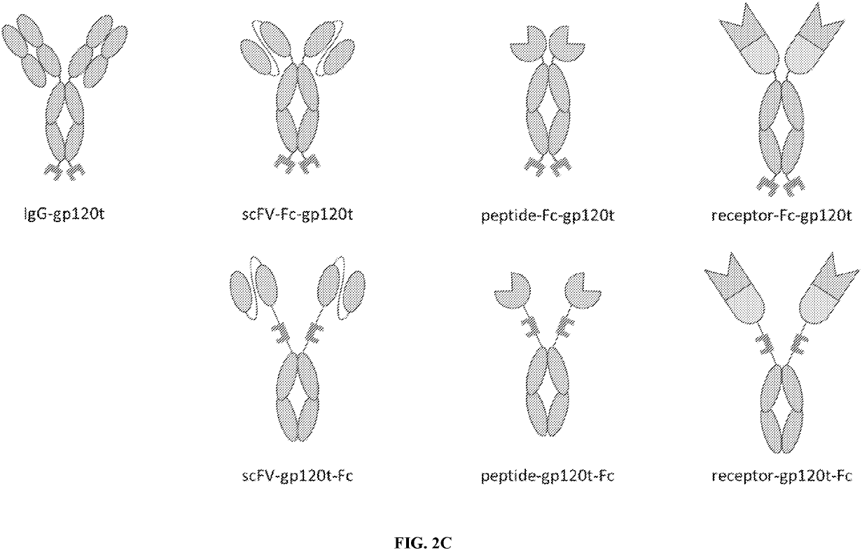

The bridging protein may comprise a truncated gp120 peptide fused or conjugated to a protein domain that binds to the target antigen of interest. In one example, the bridging protein may comprise a truncated gp120 peptide, a human Fc region, a protein domain that binds to the target antigen of interest, and one or more linker sequence. In one exemplary embodiment, the bridging protein may comprise, from N-terminus to C-terminus or from C-terminus or N-terminus, the ACE2t portion of ACE2, which is the portion of the ACE2 extracellular domain that contains all three domains required for CoV binding, a human Fc domain, and a truncated gp120 peptide, with each domain being separated by a linker (FIG. 2A). In another exemplary embodiment, the bridging protein may comprise, from N-terminus to C-terminus or from C-terminus or N-terminus, the ACE2t portion of ACE2, which is the portion of the ACE2 extracellular domain that contains all three domains required for CoV binding, a truncated gp120 peptide, and a human Fc domain, with each domain being separated by a linker (FIG. 2A). The bridging protein will be present as a homodimer due to the interaction between the Fc domains.

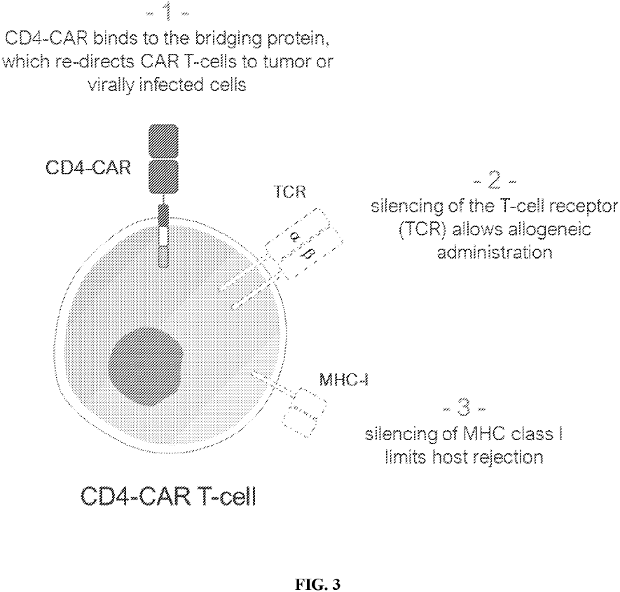

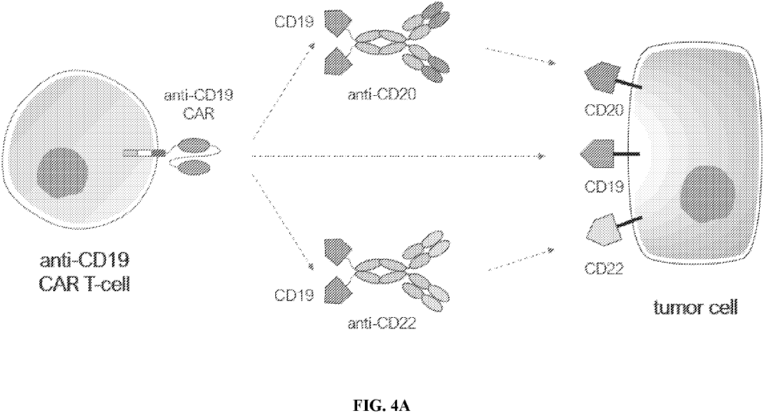

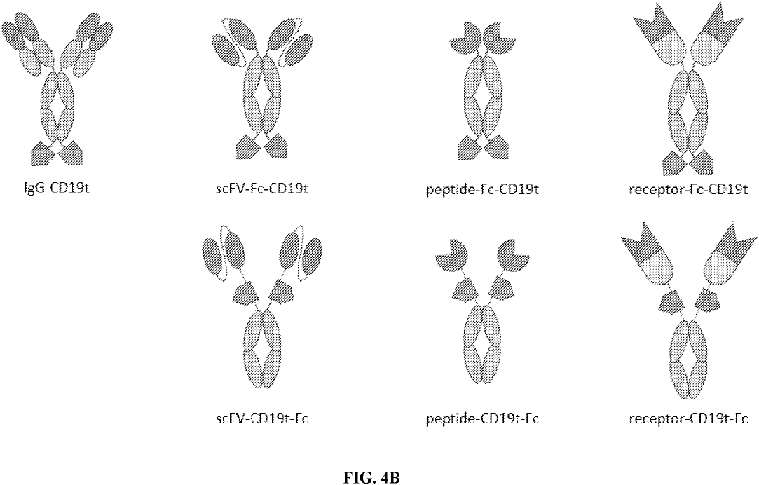

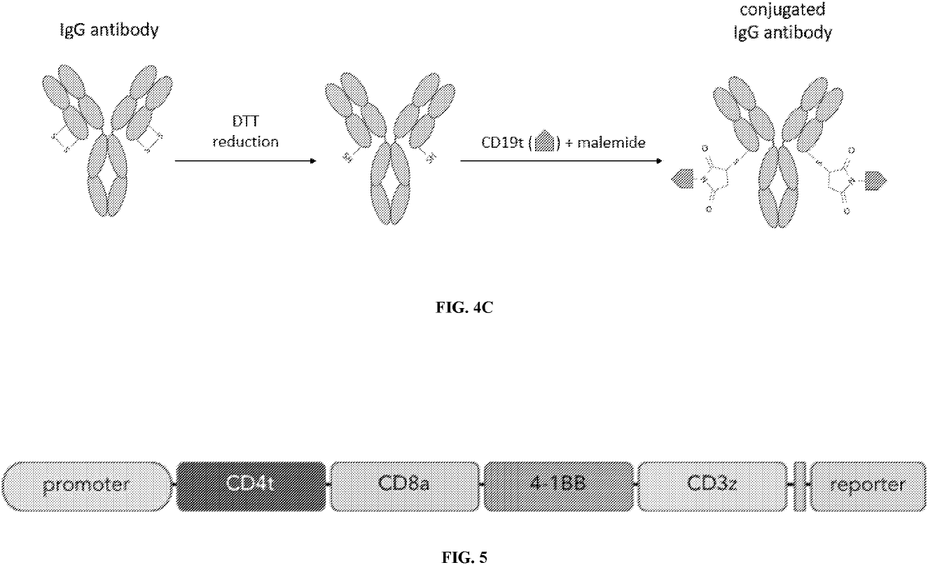

The bridging protein will re-direct the CD4-CAR T cells to recognize and kill cells expressing the antigen of interest, e.g., a CoV spike protein (FIG. 1C). The CD4-CAR T cells may have their endogenous TCR and/or MHC genes silenced to prevent allo-reactivity (FIG. 3). The CD4-CAR T cells may further have one or more inhibitory receptors (e.g., PD1 and/or TIM3) silenced to enable the T cells to persist and provide a longer lasting therapeutic effect (FIG. 3). These T cells can be prepared from healthy donor cells, making it an “off-the-shelf” solution that (a) can be rapidly provided to patients, (b) is uncompromised by the underlying disease (T cells from CoV-infected patients are severely exhausted), and (c) cost-effective (>100 doses prepared from a single donor unit) (FIG. 3).

In a further aspect, a bridging protein of the embodiments can be used to re-target other types of CAR-expressing effector cells, such as CD19 CAR T-cells. CD19 antigen loss is often encountered in patients receiving anti-CD19 CAR T-cell therapy, leading to disease relapse. Simultaneous or sequential administration of bridge proteins can allow for methods to re-direct anti-CD19 CAR T cells to other antigens on malignant cells. Thus, such methods allow for the treatment of otherwise refractory disease.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

“Nucleic acid,” “nucleic acid sequence,” “oligonucleotide,” “polynucleotide” or other grammatical equivalents as used herein means at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

The terms “peptide,” “polypeptide” and “protein” used herein refer to polymers of amino acid residues. These terms also apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers. In the present case, the term “polypeptide” encompasses an antibody or a fragment thereof.

As used herein, a “safe harbor” profile refers to the insertion of foreign genetic material into the genome of engineered cells at sites where transgene expression is sustained (i.e., not silenced) and does not disrupt expression of endogenous genes. For example, a “genetically safe harbor profile” may refer to a transgenic event that is positioned outside of the coding and expression control regions of endogenous genes. In some aspects, identifying whether an engineered cell has a safe harbor profile may comprise performing whole genome sequencing or integration site analysis.

### II. Bridging Proteins

The bridging protein comprises a CAR-binding domain and a protein domain that binds to the target antigen of interest. In some cases, the CAR-binding domain and the protein domain that binds to the target antigen of interest may be a chemical fusion of the two domains. The arrangement could be multimeric, such as a diabody or multimers. The multimers are most likely formed by cross pairing of the variable portion of the light and heavy chains into a diabody.

In some embodiments, the bridging protein comprises a CAR-binding domain, an antigen-binding domain, and, optionally, one or more linker sequence. In some cases, a linker is present between the CAR-binding domain and the antigen-binding domain. In some cases, the CAR-binding domain is directly fused to the antigen-binding domain.

In some embodiments, the bridging protein comprises a CAR-binding domain, a human Fc region, an antigen-binding domain, and, optionally, one or more linker sequence. In one embodiment, the bridging protein may comprise, from N-terminus to C-terminus or from C-terminus or N-terminus, an antigen-binding domain, a human Fc domain, and a CAR-binding domain, with each domain either being separated by a linker or being directly fused (FIGS. 2A-2C). In another embodiment, the bridging protein may comprise, from N-terminus to C-terminus or from C-terminus or N-terminus, an antigen-binding domain, a CAR-binding domain, and a human Fc domain, with each domain either being separated by a linker or being directly fused (FIGS. 2A-2C). The bridging protein may be present as a homodimer due to the presence of disulfide bonds formed between the Fc domains . However, in any of the provided embodiments, the bridging protein may be a monomer.

### A. CAR-Binding Domain

The bridging proteins comprise a CAR-binding domain. The CAR-binding domain is a protein domain that is sufficient to interact with the CAR expressed by the CAR-T cells whose effector functions are sought to be redirected. The CAR-binding domain may be positioned either between the Fc domain and the antigen-binding domain, or the CAR-binding domain may be positioned at either terminal end of the bridging protein. The CAR-binding domain may comprise the antigen-binding portions of an antibody, or antibody fragment, that specifically recognizes the CAR. In cases where the CAR comprises a ligand as its targeting domain, the CAR-binding domain of the bridging protein may comprise a portion of a receptor that binds the ligand. In cases where the CAR comprises a receptor as its targeting domain, the CAR-binding domain of the bridging protein may comprise a portion of a ligand that binds the receptor. For example, if the CAR comprises a CD4 domain as its targeting domain, the CAR-binding domain of the bridging protein may comprise a gp120 domain. For example, the gp120 domain may be a truncated gp120 domain as shown in SEQ ID NO: 6, which is an 11 amino acid segment of the gp120 extracellular domain that efficiently binds to CD4. As another example, if the CAR comprises an anti-CD19 domain as its targeting domain, the CAR-binding domain of the bridging protein may comprise at least a portion of CD19, sufficient to be bound by the anti-CD19 domain of the CAR (FIG. 1E).

### B. Fc Domain

The bridging proteins may comprise an Fc domain. The Fc domain may be position either between the CAR-binding domain and the antigen-binding domain, or the Fc domain may be positioned at either terminal end of the bridging protein. In some embodiments, the Fc domain may be a human Fc domain sequence. The Fc domain may be a human heavy chain Fc domain sequence. The Fc domain may contain only the CH2 and CH3 regions of a human heavy chain Fc domain. The Fc domain may contain substitutions that prevent Fc binding to FcgR receptors to reduce the risk of non-specific targeting of the CAR T cell’s effector functions. For example, the Fc domain may comprise D265A and/or N297A substitutions, which correspond to positions 46 and 78 in SEQ ID NO: 4, respectively. In some aspects, the Fc domain has a sequence that is at least 75%, 80%, 85%, 90%, 91%, 92%, 93%, 94%, 95%, 96%, 97%, 98%, or 99% identical to the sequence provided in SEQ ID NO: 4. In some aspects, the Fc domain has a sequence that is about 75%, 80%, 85%, 90%, 91%, 92%, 93%, 94%, 95%, 96%, 97%, 98%, or 99% identical to the sequence provided in SEQ ID NO: 4. In some aspects, the Fc domain has a sequence that is identical to the sequence provided in SEQ ID NO: 4. In some aspects, the Fc domain is encoded by a codon-optimized nucleic acid. In some aspects, the Fc domain is encoded by a sequence that is at least 75%, 80%, 85%, 90%, 91%, 92%, 93%, 94%, 95%, 96%, 97%, 98%, or 99% identical to the sequence provided in SEQ ID NO: 3. In some aspects, Fc domain is encoded by a sequence that is about 75%, 80%, 85%, 90%, 91%, 92%, 93%, 94%, 95%, 96%, 97%, 98%, or 99% identical to the sequence provided in SEQ ID NO: 3. In some aspects, the Fc domain is encoded by a sequence that is identical to the sequence provided in SEQ ID NO: 3.

### C. Antigen-Binding Domain

The bridging proteins comprise an antigen-binding domain that is capable of binding to any antigen of interest. The antigen-binding domain may be positioned either between the CAR-binding domain and the Fc domain, or the antigen-binding domain may be positioned at either terminal end of the bridging protein. The antigen-binding domain may comprise the antigen-binding portions of an antibody, or antibody fragment, that specifically recognizes the antigen. An antigen-binding fragment of an antibody refers to a portion of a protein that is capable of binding specifically to an antigen. In certain embodiments, the antigen-binding fragment is derived from an antibody comprising one or more CDRs, or any other antibody fragment that binds to an antigen but does not comprise an intact native antibody structure. In certain embodiments, the antigen-binding fragment is not derived from an antibody but rather is derived from a receptor. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a single domain antibody (sdAb), a camelid antibody or a nanobody, a domain antibody, and a bivalent domain antibody.

In cases where the antigen is a ligand, the antigen-binding domain of the bridging protein may comprise a portion of a receptor that binds the ligand (FIG. 2C). In cases where the antigen is a receptor, the antigen-binding domain of the bridging protein may comprise a portion of a ligand that binds the receptor. For example, if the antigen is a CoV spike protein, the antigen-binding domain of the bridging protein may comprise the ACE2 extracellular domain. In some aspects, the ACE2 extracellular domain may be a truncated portion of the ACE2 extracellular domain (ACE2t). The ACE2t portion of the ACE2 extracellular domain may not include the proximal end of the native ACE2 extracellular domain, which contains ADAM17, TMPRSS11d, and TMPRSS2 cleavage sites used for creating the soluble form of ACE2 and facilitating CoV infection. Excluding the protease cleavage sites prevents the unintended cleavage of the bridging protein.

In certain embodiments, the antigen-binding domain can comprise a peptide (e.g., the extracellular domain of ACE2) that binds to a receptor (e.g., coronavirus spike protein). The target binding domain may comprise the ACE2t portion of the ACE2 extracellular domain. The ACE2t portion contains all three domains required for CoV binding. The ACE2t portion of the ACE2 extracellular domain does not include the proximal end of the native ACE2 extracellular domain, which contains two cleavage sites important for creating the soluble form of ACE2 and facilitating CoV infection.

In some aspects, the ACE2t portion of the ACE2 extracellular domain has a sequence that is at least 75%, 80%, 85%, 90%, 91%, 92%, 93%, 94%, 95%, 96%, 97%, 98%, or 99% identical to the sequence provided in SEQ ID NO: 2. In some aspects, the ACE2t portion of the ACE2 extracellular domain has a sequence that is about 75%, 80%, 85%, 90%, 91%, 92%, 93%, 94%, 95%, 96%, 97%, 98%, or 99% identical to the sequence provided in SEQ ID NO: 2. In some aspects, the ACE2t portion of the ACE2 extracellular domain has a sequence that is identical to the sequence provided in SEQ ID NO: 2.

In some aspects, the ACE2t portion of the ACE2 extracellular domain is encoded by a codon-optimized nucleic acid. In some aspects, the ACE2t portion of the ACE2 extracellular domain is encoded by a sequence that is at least 75%, 80%, 85%, 90%, 91%, 92%, 93%, 94%, 95%, 96%, 97%, 98%, or 99% identical to the sequence provided in SEQ ID NO: 1. In some aspects, the ACE2t portion of the ACE2 extracellular domain is encoded by a sequence that is about 75%, 80%, 85%, 90%, 91%, 92%, 93%, 94%, 95%, 96%, 97%, 98%, or 99% identical to the sequence provided in SEQ ID NO: 1. In some aspects, the ACE2t portion of the ACE2 extracellular domain is encoded by a sequence that is identical to the sequence provided in SEQ ID NO: 1.

Other exemplary antigens include surface antigens on cancer cells (FIG. 1D) and surface antigens on infected cells. The surface antigen on cancer cells may be a tumor-specific antigen, i.e., an antigen that is expressed exclusively on tumor cells. The surface antigen on cancer cells may be a tumor-associated antigen, i.e., an antigen that is expressed on healthy cells but is over-expressed on tumor cells. Examples of surface antigens on cancer cells include HER-3, Her1/HER-3 fusion; CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1, sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). Examples of surface antigens on infected cells include viral spike or envelope proteins (e.g., HIV-1 gp120, HIV-1 gp41, HIV-1 gp160, SARS-CoV S protein, SARS-CoV-2 S protein, MERS S protein, Ebolavirus glycoprotein, influenza haemaglutinin, influenza neuraminidase, hepatitis C E1, hepatitis C E2, Dengue virus E dimer, Chikungunya virus E1, Chikungunya virus E1, cytomegalovirus glycoprotein, herpes simplex virus gB, herpes simplex virus gH, herpes simplex virus gL, herpes simplex virus gM, Epstein-Barr virus gp350, and Epstein-Barr virus gp42).

### D. Linkers

The bridging proteins may comprise at least one peptide linker (or spacer) positioned between the fused polypeptide sequences, so as to allow correct folding and/or prevent steric hindrance of the fused domains. The peptide linkers may be flexible linkers. In some aspects, a linker is between 2 and 20 peptides long, between 2 and 18 peptides long, between 2 and 16 peptides long, between 2 and 14 peptides long, between 2 and 12 peptides long, between 2 and 10 peptides long, between 4 and 20 peptides long, between 4 and 18 peptides long, between 4 and 16 peptides long, between 4 and 14 peptides long, between 4 and 12 peptides long, or between 4 and 10 peptides long. In some aspects, a linker comprises a core sequence of GGGS (SEQ ID NO: 7). In some aspects, a linker comprises the sequence SSGGGGSGGGGGGSS (SEQ ID NO: 9) or the sequence SSGGGGSGGGGGGSSRSS (SEQ ID NO: 10). Preferably, a linker comprises the sequence SSGGGGS (SEQ ID NO: 8). Where a bridging protein contains more than one linker, each linker in the bridging protein may have the same sequence or each linker may have a different sequence.

Alternatively, the CAR-binding domain and the antigen-binding domain of the bridging proteins may be chemically conjugated. For example, cysteine residues of the antigen-binding domain may be site-specifically and efficiently coupled with a thiol-reactive reagent. The thiol-reactive agent may be, for example, a maleimide, an iodoacetamide, a pyridyl disulfide, or other thiol-reactive conjugation partner. As such, the CAR-binding domain portion of the bridging protein may comprise, for example, a maleimide loop. Chemical conjugation can then be initiated with dithiothreitol (DTT) reduction and the addition of the CAR-binding domain-maleimide.

### III. Chimeric Antigen Receptors

Chimeric antigen receptor (CAR) molecules are recombinant fusion proteins and are distinguished by their ability to both bind a target (e.g., a coronavirus spike protein) and transduce activation signals via immunoreceptor activation motifs (ITAMs) present in their cytoplasmic tails in order to activate genetically modified immune effector cells for killing, proliferation, and cytokine production.

A chimeric antigen receptor according to the embodiments can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, site-directed mutagenesis, etc.). The resulting coding region can be inserted into an expression vector and used to transform suitable host allogeneic or autologous immune effector cells.

Embodiments of the CARs described herein include nucleic acids encoding a target-specific chimeric antigen receptor (CAR) polypeptide comprising an intracellular signaling domain, a transmembrane domain, and an extracellular domain comprising a target-binding domain. Optionally, a CAR can comprise a hinge domain positioned between the transmembrane domain and the target-binding domain. In certain aspects, a CAR of the embodiments further comprises a signal peptide that directs expression of the CAR to the cell surface. For example, in some aspects, a CAR can comprise a signal peptide from GM-CSF.

In certain embodiments, the CAR can also be co-expressed with a membrane-bound cytokine to improve persistence when there is a low amount of target. For example, CAR can be co-expressed with membrane-bound IL-15.

Depending on the arrangement of the domains of the CAR and the specific sequences used in the domains, immune effector cells expressing the CAR may have different levels activity against target cells. In some aspects, different CAR sequences may be introduced into immune effector cells to generate engineered cells, the engineered cells selected for elevated SRC and the selected cells tested for activity to identify the CAR constructs predicted to have the greatest therapeutic efficacy.

The chimeric construct may be introduced into immune effector cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression. Alternatively, a viral vector (e.g., a retroviral vector, adenoviral vector, adeno-associated viral vector, or lentiviral vector) can be used to introduce the chimeric construct into immune effector cells. Suitable vectors for use in accordance with the method of the present invention are non-replicating in the immune effector cells. A large number of vectors are known that are based on viruses, where the copy number of the virus maintained in the cell is low enough to maintain the viability of the cell, such as, for example, vectors based on HIV, SV40, EBV, HSV, or BPV.

### A. Antigen-Binding Domain

In certain embodiments, an antigen binding domain can comprise complementarity determining regions of a monoclonal antibody, variable regions of a monoclonal antibody, and/or antigen binding fragments thereof. For example, the antigen binding domain may comprise the complementarity determining regions of an antibody that binds to CD19. A “complementarity determining region (CDR)” is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor contains three CDRs (CDR1, CDR2, and CDR3). Since the antigen receptors are typically composed of two polypeptide chains, there are six CDRs for each antigen receptor that can come into contact with the antigen — each heavy and light chain contains three CDRs. Because most sequence variation associated with immunoglobulins and T-cell receptors are found in the CDRs, these regions are sometimes referred to as hypervariable domains. Among these, CDR3 shows the greatest variability as it is encoded by a recombination of the VJ (VDJ in the case of heavy chain and TCR αβ chain) regions. In another embodiment, that specificity is derived from a peptide (e.g., cytokine) that binds to a receptor. In another embodiment, that specificity is derived from a receptor (e.g., the extracellular domain of CD4, such as the D1 and D2 domains of CD4) that binds to a viral glycoprotein. In aspects where the antigen-binding domain is derived from CD4, the portions of CD4 that form the antigen-binding domain may be mutated to limit binding to MHC Class II.

It is contemplated that the CAR nucleic acids, in particular the scFv sequences are human genes to enhance cellular immunotherapy for human patients. In a specific embodiment, there is provided a full-length CAR cDNA or coding region. The antigen binding regions or domains can comprise a fragment of the VH and VL chains of a single-chain variable fragment (scFv) derived from a particular mouse, or human or humanized monoclonal antibody. The fragment can also be any number of different antigen binding domains of an antigen-specific antibody. In a more specific embodiment, the fragment is an antigen-specific scFv encoded by a sequence that is optimized for human codon usage for expression in human cells. In certain aspects, VH and VL domains of a CAR are separated by a linker sequence, such as a Whitlow linker. CAR constructs that may be modified or used according to the embodiments are also provided in International (PCT) Patent Publication No. WO2015/123642, incorporated herein by reference.

As previously described, the prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the LCDR1-3 sequences and the HCDR1-3 sequences of an antibody that binds to an antigen of interest, such as tumor associated antigen. In further aspects, however, two of more antibodies that bind to an antigen of interest are identified and a CAR is constructed that comprises: (1) the HCDR1-3 sequences of a first antibody that binds to the antigen; and (2) the LCDR1-3 sequences of a second antibody that binds to the antigen. Such a CAR that comprises HCDR and LCDR sequences from two different antigen binding antibodies may have the advantage of preferential binding to particular conformations of an antigen (e.g., conformations preferentially associated with cancer cells versus normal tissue).

Alternatively, it is shown that a CAR may be engineered using VH and VL chains derived from different mAbs to generate a panel of CAR+ T cells. The antigen binding domain of a CAR can contain any combination of the LCDR1-3 sequences of a first antibody and the HCDR1-3 sequences of a second antibody.

### B. Hinge Domain

In certain aspects, a CAR polypeptide of the embodiments can include a hinge domain positioned between the target-binding domain and the transmembrane domain. In some cases, a hinge domain may be included in CAR polypeptides to provide adequate distance between the target-binding domain and the cell surface or to alleviate possible steric hindrance that could adversely affect target binding or effector function of CAR-modified T cells. The hinge domain may comprise a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor.

In some cases the CAR hinge domain could be derived from human immunoglobulin (Ig) constant region or a portion thereof including the Ig hinge, or from human CD8a transmembrane domain (FACDIYIWAPLAGTCGVLLLSLVITLYCNHRN; SEQ ID NO: 11) and CD8a-hinge region (KPTTTPAPRPPTPAPTIASQPLSLRPEACRPAAGGAVHTRGLD; SEQ ID NO: 12). In one aspect, the CAR hinge domain can comprise a hinge—CH₂—CH₃ region of antibody isotype IgG₄ (ESKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLPSS IEKTISKAKGQPREPQVYTLPPSQEEMTKNQVSLTCLVKGFYPSDIAVEWESNGQPEN NYKTTPPVLDSDGSFFLYSRLTVDKSRWQEGNVFSCSVMHEALHNHYTQKSLSLSL GKM; SEQ ID NO: 13). In some aspects, point mutations could be introduced in antibody heavy chain CH₂ domain to reduce glycosylation and non-specific Fc gamma receptor binding of CAR-modified immune effector cells.

In certain aspects, a CAR hinge domain of the embodiments comprises an Ig Fc domain that comprises at least one mutation relative to wild type Ig Fc domain that reduces Fc-receptor binding. For example, the CAR hinge domain can comprise an IgG4-Fc domain that comprises at least one mutation relative to wild type IgG4-Fc domain that reduces Fc-receptor binding. In some aspects, a CAR hinge domain comprises an IgG4-Fc domain having a mutation (such as an amino acid deletion or substitution) at a position corresponding to L235 and/or N297 relative to the wild type IgG4-Fc sequence. For example, a CAR hinge domain can comprise an IgG4-Fc domain having a L235E and/or a N297Q mutation relative to the wild type IgG4-Fc sequence. In further aspects, a CAR hinge domain can comprise an IgG4-Fc domain having an amino acid substitution at position L235 for an amino acid that is hydrophilic, such as R, H, K, D, E, S, T, N or Q or that has similar properties to an “E,” such as D. In certain aspects, a CAR hinge domain can comprise an IgG4-Fc domain having an amino acid substitution at position N297 for an amino acid that has similar properties to a “Q,” such as S or T.

### C. Transmembrane Domain

The target-specific extracellular domain and the intracellular signaling-domain may be linked by a transmembrane domain. Polypeptide sequences that can be used as part of transmemebrane domain include, without limitation, the human CD4 transmembrane domain, the human CD28 transmembrane domain, the transmembrane human CD3ξ domain, or a cysteine mutated human CD3ξ domain, or other transmembrane domains from other human transmembrane signaling proteins, such as CD16, CD8, and erythropoietin receptor. In some aspects, for example, the transmembrane domain may comprise a sequence at least 85%, 90%, 91%, 92%, 93%, 94%, 95%, 96%, 97%, 98%, 99% or 100% identical to one of those provided in U.S. Pat. Publication No. 2014/0274909 (e.g. a CD8 and/or a CD28 transmembrane domain) or U.S. Pat. No. 8,906,682 (e.g. a CD8α transmembrane domain), both incorporated herein by reference on their entirety. In certain specific aspects, transmembrane regions may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain.

### D. Intracellular Signaling Domain

The intracellular signaling domain of the chimeric antigen receptor of the embodiments is responsible for activation of at least one of the normal effector functions of the immune cell engineered to express a CAR. The term “effector function” refers to a specialized function of a differentiated cell. Effector function of a T cell, for example, may be cytolytic activity or helper activity including the secretion of cytokines. Effector function in a naive, memory, or memory-type T cell includes antigen-dependent proliferation. Thus, the term “intracellular signaling domain” refers to the portion of a protein that transduces the effector function signal and directs the cell to perform a specialized function. In some aspects, the intracellular signaling domain is derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ξ and CD28, CD27, 4-1BB/CD137, ICOS/CD278, IL-2Rβ/CD122, IL-2Rα/CD132, DAP10, DAP12, CD40, OX40/CD134, and combinations thereof, as well as other similar molecules and fragments. Intracellular signaling portions of other members of the families of activating proteins can be used, such as FcyRIII and FcsRI.

While usually the entire intracellular signaling domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. One or multiple cytoplasmic domains may be employed, as so-called third generation CARs have at least two or three signaling domains fused together for additive or synergistic effect, for example the CD28 and 4-1BB can be combined in a CAR construct. In certain specific aspects, the intracellular signaling domain comprises a sequence 85%, 90%, 91%, 92%, 93%, 94%, 95%, 96%, 97%, 98%, 99% or 100% identical to a CD3ξ intracellular domain (RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR; SEQ ID NO: 14), a CD28 intracellular domain, a CD137 intracellular domain, or a domain comprising a CD28 intracellular domain fused to the 4-1BB intracellular domain. In a preferred embodiment, the human CD3ξ intracellular domain is used as the intracellular signaling domain for a CAR of the embodiments.

In specific embodiments, intracellular receptor signaling domains in the CAR include those of the T cell antigen receptor complex, such as the ξ chain of CD3, also Fcγ RIII costimulatory signaling domains, CD28, CD27, DAP10, CD137, OX40, CD2, alone or in a series with CD3ξ, for example. In specific embodiments, the intracellular domain (which may be referred to as the cytoplasmic domain) comprises part or all of one or more of TCRξ chain, CD28, CD27, OX40/CD134, 4-1BB/CD137, FcεRIγ, ICOS/CD278, IL-2Rβ/CD122, IL,-2Rα/CD132, DAP10, DAP12, and CD40. In some embodiments, one employs any part of the endogenous T-cell receptor complex in the intracellular domain. One or multiple cytoplasmic domains may be employed, as so-called third generation CARs have at least two or three signaling domains fused together for additive or synergistic effect, for example.

In some embodiments, the CAR comprises additional other costimulatory domains. Other costimulatory domains can include, but are not limited to one or more of CD28, CD27, OX-40 (CD134), DAP10, and 4-1BB (CD137). In addition to a primary signal initiated by CD3ξ, an additional signal provided by a human costimulatory receptor inserted in a human CAR is important for full activation of T cells and could help improve in vivo persistence and the therapeutic success of the adoptive immunotherapy.

### IV. Modification of Endogenous Gene Expression

In some aspects, the engineered immune effector cells are modified to decrease or eliminate the expression of one or more endogenous genes. For example, the engineered immune effector cells may be modified to knock down or knock out at least one immune checkpoint protein. The at least one immune checkpoint gene may be selected from the group consisting of: PD1, CTLA4, LAG3, TIM3, TIGIT, CD96, BTLA, KIRs, adenosine A2a receptor, Vista, IDO, FAS, SIRP alpha, CISH, SHP-1, FOXP3, LAIR1, PVRIG, PPP2CA, PPP2CB, PTPN6, PTPN22, CD160, CRTAM, SIGLEC7, SIGLEC9, CD244, TNFRSF10B, TNFRSF10A, CASP8, CASP10, CASP3, CASP6, CASP7, FADD, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, PRDM1, BATF, GUCY1A2, GUCY1A3, GUCY1B2, and GUCY1B3.

In some aspects, the engineered immune effector cells are modified to decrease or eliminate the expression of one or more HIV co-receptor. For example, the engineered immune effector cells are modified such that CCR5 expression is silenced.

As another example, HLA genes in the engineered immune effector cells may be modified in various ways. For example, the engineered immune effector cells may be engineered such that they do not express functional HLA-A, HLA-B, and/or HLA-C on their surface. The HLA-A negative engineered immune effector cells may be derived from an HLA-homozygous individual. Alternatively, the engineered immune effector cells may be HLA-A homozygous. Further, the engineered immune effector cells, regardless of whether they are HLA-A negative or HLA-A homozygous, may be HLA-homozygous at HLA-B, HLA-C, and/or HLA-DRB1 alleles.

In some aspects, the engineered immune effector cells may be modified to knock down or knock out the expression of one or more T-cell receptor component. For example, in some aspects, the cell lacks expression or have reduced expression of TCRα, TCRβ, TCRα and TCRβ, TCRγ, TCRδ, TCRγ and TCRδ, or any combination of the foregoing. Such can occur by any suitable manner, including by introducing zinc finger nucleases (ZFN), for example, targeting the constant region of one or more of the TCR receptor components.

Knocking out an endogenous gene may comprise introducing into the cells an artificial nuclease that specifically targets the endogenous gene’s locus. In various aspects, the artificial nuclease may be a zinc finger nuclease, TALEN, or CRISPR/Cas9. In various aspects, introducing into the cells an artificial nuclease may comprise introducing mRNA encoding the artificial nuclease into the cells.

For example, in some aspects, a target endogenous gene includes a deletion or mutation generated by a zinc finger nuclease, TALEN, or CRISPR/Cas9 system that renders the gene or gene product non-functional. Such a deletion or mutation may occur in both alleles of the target endogenous gene.

Knocking down the expression of an endogenous gene may comprise introducing into the cells an inhibitory nucleic acid, such as a construct encoding a miRNA. An inhibitory nucleic acid may inhibit the transcription of a gene or prevent the translation of a gene transcript in a cell. An inhibitory nucleic acid may be from 16 to 1000 nucleotides long, and in certain embodiments from 18 to 100 nucleotides long. In certain embodiments, the inhibitory nucleic acid is an isolated nucleic acid that binds or hybridizes to a gene of interest. The inhibitory nucleic acid may silence the expression of a target gene by at least 30%, 35%, 40%, 45%, 50%, 55%, 60%, 65%, 70%, 75%, 80%, 85%, 90%, or 95%, and preferably by at least 75%.

Inhibitory nucleic acids are well known in the art. For example, siRNA, shRNA, miRNA and double-stranded RNA have been described in U.S. Pat.s 6,506,559 and 6,573,099, as well as in U.S. Pat. Publications 2003/0051263, 2003/0055020, 2004/0265839, 2002/0168707, 2003/0159161, and 2004/0064842, all of which are herein incorporated by reference in their entirety. In various aspects, knocking down the expression of an endogenous gene may comprise the use of miRNA expression constructs, of multiple miRNAs and use thereof to knockdown target gene expression. In some aspects, the expression constructs include a promoter element, a spacer sequence and a miRNA coding sequence. Examples of such miRNA expression constructs can be found in WO 2019/186274 and U.S. Pat. 9,556,433, which are each incorporated herein by reference in their entirety.

Within certain aspects expression vectors are employed to express a nucleic acid of interest, such as a nucleic acid that inhibits the expression of a particular gene. Expression requires that appropriate signals be provided in the vectors, and which include various regulatory elements, such as enhancers/promoters from both viral and mammalian sources that drive expression of the genes of interest in host cells. Elements designed to optimize RNA stability in host cells also are defined. The conditions for the use of a number of dominant drug selection markers for establishing permanent, stable cell clones expressing the products are also provided, as is an element that links expression of the drug selection markers to expression of the polypeptide.

### A. Regulatory Elements

Throughout this application, the term “expression construct” or “expression vector” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest i.e., as is the case with RNA molecules of the embodiments.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for eukaryotic RNA polymerase (Pol) I, II or III. Much of the thinking about how promoters are organized derives from analyses of several viral Pol II promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In some embodiments, the promoter comprises an Elongation Factor 1 short (EF1s) promoter. In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof. In some aspects, a promoter for use according to the instant embodiments is a non-tissue specific promoter, such as a constitutive promoter.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene or miRNA of interest in an expression construct (Table 1 and Table 2). Additionally, any promoter/enhancer combination (as per the Eukaryotic Promoter Data Base EPDB) could also be used to drive expression of the gene or miRNA of interest. Truncated promoters may also be used to drive expression. Eukaryotic cells can support cytoplasmic transcription from certain bacterial promoters if the appropriate bacterial polymerase is provided, either as part of the delivery complex or as an additional genetic expression construct.

TABLE 1

Promoter and/or Enhancer

| | |
|---|---|
| Promoter/Enhancer | References |
| Elongation Factor 1 alpha (EF1α) | Kim et al., 1990 |
| Immunoglobulin Heavy Chain | Banerji et al., 1983; Gilles et al., 1983; Grosschedl et al., 1985; Atchinson et al., 1986; Imler et al., 1987; Weinberger et al., 1988; Kiledjian et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et al.; 1990 |
| HLA DQ a and/or DQ β | Sullivan et al., 1987 |
| β-Interferon | Goodbourn et al., 1986; Fujita et al., 1987; Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| P-Globin | Trudel et al., 1987 |
| c-fos | Cohen etal., 1987 |
| c-HA-ras | Triesman, 1986; Deschamps et al., 1985 |
| Insulin | Edlund et al., 1985 |
| Neural Cell Adhesion Molecule (NCAM) | Hirsh et al., 1990 |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins (GRP94 and GRP78) | Chang et al., 1989 |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor (PDGF) | Pech et al., 1989 |
| Duchenne Muscular Dystrophy | Klamut et al., 1990 |
| SV40 | Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988 |
| Polyoma | Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell and Villarreal, 1988 |
| Retroviruses | Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989 |
| Papilloma Virus | Campo et al., 1983; Lusky et al., 1983; Spandidos and/or Wilkie, 1983; Spalholz et al., 1985; Lusky et al., 1986; Cripe et al., 1987; Gloss et al., 1987; Hirochika et al., 1987; Stephens et al., 1987 |
| Hepatitis B Virus | Bulla et al., 1988; Jameel et al., 1986; Shaul et al., 1987; Spandau et al., 1988; Vannice et al., 1988 |
| Human Immunodeficiency Virus | Muesing et al., 1987; Hauber et al., 1988; Jakobovits et al., 1988; Feng et al., 1988; Takebe et al., 1988; Rosen et al., 1985; Berkhout et al., 1989; Laspia et al., 1989; Sharp et al., 1989; Braddock et al., 1989 |
| Cytomegalovirus (CMV) | Weber et al., 1984; Boshart et al., 1985; Foecking et al., 1986 |
| Gibbon Ape Leukemia Virus | Holbrook et al., 1987; Quinn et al., 1989 |

TABLE 2

Inducible Elements

| | | |
|---|---|---|
| Element | Inducer | References |
| MT II | Phorbol Ester (TFA) Heavy metals | Palmiter et al., 1982; Haslinger et al., 1985; Searle et al., 1985; Stuart et al., 1985; Imagawa et al., 1987, Karin et al., 1987; Angel et al., 1987b; McNeall et al., 1989 |
| MMTV (mouse mammary tumor virus) | Glucocorticoids | Huang et al., 1981; Lee et al., 1981; Majors et al., 1983; Chandler et al., 1983; Ponta et al., 1985; Sakai et al., 1988 |
| β-Interferon | poly(rI)x poly(rc) | Tavernier et al., 1983 |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle Disease Virus | Hug et al., 1988 |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T Antigen | Taylor et al., 1989, 1990a, 1990b |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

Where any cDNA insert is employed, one will typically include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. In some aspects, however, a polyadenylation signal sequence is not included in a vector of the embodiments. For example, incorporation of such a signal sequence in lentiviral vectors (before a 3′ LTR) can reduce resulting lentiviral titers.

A spacer sequence may be included in the nucleic acid construct. The presence of a spacer appears to enhance knockdown efficiency of miRNA (Stegmeier et al., 2005). Spacers may be any nucleotide sequence. In some aspects, the spacer is GFP.

Also contemplated as an element of the expression cassette is a terminator. These elements can serve to enhance message levels and to minimize read through from the cassette into other sequences.

### B. Selectable Markers

In certain embodiments of the invention, the cells contain nucleic acid constructs of the present invention, a cell may be identified in vitro, ex vivo or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

### V. Delivery of Nucleic Acid Molecules and Expression Vectors

In certain aspects, vectors for delivery of nucleic acids of the embodiments could be constructed to express these factors in cells. In a particular aspect, the following systems and methods may be used in delivery of nucleic acids to desired cell types.

### A. Homologous Recombination

In certain aspects of the embodiments, the vectors encoding nucleic acid molecules of the embodiments may be introduced into cells in a specific manner, for example, via homologous recombination. Current approaches to express genes in stem cells have involved the use of viral vectors (e.g., lentiviral vectors) or transgenes that integrate randomly in the genome. These approaches have not been successful due in part because the randomly integrated vectors can activate or suppress endogenous gene expression, and/or the silencing of transgene expression. The problems associated with random integration could be partially overcome by homologous recombination to a specific locus in the target genome.

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid 1980s. The process involves several steps of physical breaking and the eventual rejoining of DNA. This process is most widely used in nature to repair potentially lethal double-strand breaks in DNA. In addition, homologous recombination produces new combinations of DNA sequences during meiosis, the process by which eukaryotes make germ cells like sperm and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Homologous recombination can be used as targeted genome modification. The efficiency of standard HR in mammalian cells is only 10⁻⁶ to 10⁻⁹ of cells treated (Capecchi, 1990). The use of meganucleases, or homing endonucleases, such as I-Scel have been used to increase the efficiency of HR. Both natural meganucleases as well as engineered meganucleases with modified targeting specificities have been utilized to increase HR efficiency (Pingoud and Silva, 2007; Chevalier et al., 2002). Another path toward increasing the efficiency of HR has been to engineer chimeric endonucleases with programmable DNA specificity domains (Silva et al., 2011). Zinc-finger nucleases (ZFN) are one example of such a chimeric molecule in which Zinc-finger DNA binding domains are fused with the catalytic domain of a Type IIS restriction endonuclease such as FokI (as reviewed in Durai et al., 2005; WO2005028630). Another class of such specificity molecules includes Transcription Activator Like Effector (TALE) DNA binding domains fused to the catalytic domain of a Type IIS restriction endonuclease such as FokI (Miller et al., 2011: WO2010079430).

### B. Nucleic Acid Delivery Systems

One of skill in the art would be well equipped to construct a vector through standard recombinant techniques (see, for example, Sambrook et al., 2001 and Ausubel et al., 1996, both incorporated herein by reference). Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV etc), lentiviral vectors (e.g., derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

### 1. Episomal Vectors

The use of plasmid- or liposome-based extra-chromosomal (i.e., episomal) vectors may be also provided in certain aspects of the invention, for example, for reprogramming of somatic cells. Such episomal vectors may include, e.g., oriP-based vectors, and/or vectors encoding a derivative of EBV-protein EBNA-1. These vectors may permit large fragments of DNA to be introduced to a cell and maintained extra-chromosomally, replicated once per cell cycle, partitioned to daughter cells efficiently, and elicit substantially no immune response.

In particular, EBNA-1, the only viral protein required for the replication of the oriP-based expression vector, does not elicit a cellular immune response because it has developed an efficient mechanism to bypass the processing required for presentation of its antigens on MHC class I molecules (Levitskaya et al., 1997). Further, EBNA-1 can act in trans to enhance expression of the cloned gene, inducing expression of a cloned gene up to 100-fold in some cell lines (Langle-Rouault et al., 1998; Evans et al., 1997). Finally, the manufacture of such oriP-based expression vectors is inexpensive.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi’s sarcoma herpes virus (KSHV); Herpes virus saimiri (HS) and Marek’s disease virus (MDV). Also other sources of episome-based vectors are contemplated, such as yeast ARS, adenovirus, SV40, or BPV.

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell’s nucleus or cytoplasm.

### 2. Transposon-Based System

According to a particular embodiment the introduction of nucleic acids may use a transposon - transposase system. The used transposon - transposase system could be the well known Sleeping Beauty, the Frog Prince transposon - transposase system (for the description of the latter see e.g., EP1507865), or the TTAA-specific transposon piggyback system.

Transposons are sequences of DNA that can move around to different positions within the genome of a single cell, a process called transposition. In the process, they can cause mutations and change the amount of DNA in the genome. Transposons were also once called jumping genes, and are examples of mobile genetic elements.

There are a variety of mobile genetic elements, and they can be grouped based on their mechanism of transposition. Class I mobile genetic elements, or retrotransposons, copy themselves by first being transcribed to RNA, then reverse transcribed back to DNA by reverse transcriptase, and then being inserted at another position in the genome. Class II mobile genetic elements move directly from one position to another using a transposase to “cut and paste” them within the genome.

### 3. Viral Vectors

In generating recombinant viral vectors, non-essential genes are typically replaced with a gene or coding sequence for a heterologous (or non-native) protein or nucleic acid. Viral vectors are a kind of expression construct that utilizes viral sequences to introduce nucleic acid and possibly proteins into a cell. The ability of certain viruses to infect cells or enter cells via pH-dependent or pH-independent mechanisms, to integrate their genetic cargo into a host cell genome and to express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid (i.e., the vector genome) to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Depending on the tropism of the envelope protein used to cover the vector particles surface, retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; Giry-Laterriere et al., 2011; U.S. Pat. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and that is described in U.S. Pat. 5,994,136, incorporated herein by reference.

### C. Nucleic Acid Delivery

Introduction of a nucleic acid, such as DNA or RNA, into cells to be programmed with the current invention may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Patent Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Patent Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

### 2. Liposome-Mediated Transfection

In a certain embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a nucleic acid complexed with Lipofectamine (Gibco BRL) or Superfect (Qiagen). The amount of liposomes used may vary upon the nature of the liposome as well as the cell used, for example, about 5 to about 20 µg vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

### 3. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 µg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### 4. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 5. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

### D. Cell Culturing

Generally, cells of the present invention are cultured in a culture medium, which is a nutrient-rich buffered solution capable of sustaining cell growth.

Culture media suitable for isolating, expanding and differentiating stem cells according to the method described herein include but not limited to high glucose Dulbecco’s Modified Eagle’s Medium (DMEM), DMEM/F-12, Liebovitz L-15, RPMI 1640, Iscove’s modified Dulbecco’s media (IMDM), and Opti-MEM SFM (Invitrogen Inc.). Chemically Defined Medium comprises a minimum essential medium such as Iscove’s Modified Dulbecco’s Medium (IMDM) (Gibco), supplemented with human serum albumin, human Ex Cyte lipoprotein, transferrin, insulin, vitamins, essential and non-essential amino acids, sodium pyruvate, glutamine and a mitogen is also suitable. As used herein, a mitogen refers to an agent that stimulates cell division of a cell. An agent can be a chemical, usually some form of a protein that encourages a cell to commence cell division, triggering mitosis. In one embodiment, serum free media such as those described in U.S. Pat. No. 5,908,782 and WO96/39487, and the “complete media” as described in U.S. Pat. No. 5,486,359 are contemplated for use with the method described herein. In some embodiments, the culture medium is supplemented with 10% Fetal Bovine Serum (FBS), human autologous serum, human AB serum or platelet rich plasma supplemented with heparin (2 U/mL). Cell cultures may be maintained in a CO₂ atmosphere, e.g., 5% to 12%, to maintain pH of the culture fluid, incubated at 37° C. in a humid atmosphere and passaged to maintain a confluence below 85%.

### VI. Immune Effector Cells

Immune effectors cells may be T cells (e.g., regulatory T cells, CD4+ T cells, CD8+ T cells, or gamma-delta T cells), natural killer (NK) cells, invariant NK cells, or NKT cells. Also provided herein are methods of producing and engineering the immune effector cells as well as methods of using and administering the cells for adoptive cell therapy, in which case the cells may be autologous or allogeneic. Thus, the immune effector cells may be used as immunotherapy, such as to target cancer cells.

The immune effector cells may be isolated from subjects, particularly human subjects. The immune effector cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from a blood bank. Immune effector cells can be collected, enriched, and/or purified from any tissue or organ in which they reside in the subject including, but not limited to, blood, cord blood, spleen, thymus, lymph nodes, bone marrow, tissues removed and/or exposed during surgical procedures, and tissues obtained via biopsy procedures. The isolated immune effector cells may be used directly, or they can be stored for a period of time, such as by freezing.

Tissues/organs from which the immune effector cells are enriched, isolated, and/or purified may be isolated from both living and non-living subjects, wherein the non-living subjects are organ donors. Immune effector cells isolated from cord blood may have enhanced immunomodulation capacity, such as measured by CD4- or CD8-positive T cell suppression. The immune effector cells may be isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune effector cell activity. Thus, the cells will be autologous to the subject in need of therapy. Alternatively, the population of immune effector cells can be obtained from a donor, preferably an allogeneic donor. Allogeneic donor cells may or may not be human-leukocyte-antigen (HLA)-compatible. To be rendered subject-compatible, allogeneic cells can be treated to reduce immunogenicity.

Sources of immune effector cells include both allogeneic and autologous sources. In some cases, immune effector cells may be differentiated from stem cells or induced pluripotent stem cells (iPSCs). Thus, cell for engineering according to the embodiments can be isolated from umbilical cord blood, peripheral blood, human embryonic stem cells, or iPSCs. For example, allogeneic T cells can be modified to include a chimeric antigen receptor (and optionally, to lack functional TCR and/or MHC). In some aspects, the immune effector cells are primary human T cells, such as T cells derived from human peripheral blood mononuclear cells (PBMC), PBMC collected after stimulation with G-CSF, bone marrow, or umbilical cord blood. Following transfection or transduction (e.g., with a CAR expression construct), the cells may be immediately infused or may be stored. In certain aspects, following transfection, the cells may be propagated for days, weeks, or months ex vivo as a bulk population within about 1, 2, 3, 4, 5 days or more following gene transfer into cells. In a further aspect, following transfection, the transfectants are cloned and a clone demonstrating presence of a single integrated or episomally maintained expression cassette or plasmid, and expression of the chimeric antigen receptor is expanded ex vivo. The clone selected for expansion demonstrates the capacity to specifically recognize and lyse antigen-expressing target cells. The recombinant T cells may be expanded by stimulation with IL-2, or other cytokines that bind the common gamma-chain (e.g., IL-7, IL-12, IL-15, IL-21, and others). The recombinant T cells may be expanded by stimulation with artificial antigen presenting cells. The recombinant T cells may be expanded on artificial antigen presenting cell or with an antibody, such as OKT3, which cross links CD3 on the T cell surface. Subsets of the recombinant T cells may be deleted on artificial antigen presenting cell or with an antibody, such as Campath, which binds CD52 on the T cell surface. In a further aspect, the genetically modified cells may be cryopreserved.

In further aspects, immune effector cells of the embodiment have been selected for high mitochondrial spare respiratory capacity (SRC). As used herein an “immune effector cell having high mitochondrial SRC” refers to an immune effector cell (e.g., a T-cell) having higher mitochondria activity or mitochondria number than a corresponding average immune effector cell (e.g., a T-cell). Thus, in some aspects, a cell composition of the embodiments comprises a population of immune effector cells having high mitochondrial SRC, for example a population of CAR-expressing T-cell having high mitochondrial SRC.

Immune effector cells, such as CD8⁺ T cells, with high mitochondrial SRC may exhibit enhanced survival relative to cells with lower SRC during stress conditions, such as high tumor burden, hypoxia, lack of nutrients for glycolysis, or a suppressive cytokine milieu. Moreover, immune effector cells selected for high mitochondrial SRC may retain cytotoxic activity, even under stress conditions. Accordingly, by selecting immune effector cells with high mitochondrial SRC improved cell composition for both therapy and for testing of CAR constructs can be produced.

In one aspects, transgenic immune effector cells are provided that comprise a reporter that can be used to determine the mitochondrial SRC of the transgenic effector cells. For example, transgenic cells may comprise a reporter polypeptide that is linked to a mitochondria localization signal. For example, the reporter can be a fluorescent polypeptide such an enhanced Yellow Fluorescence Protein (YFP) or an enhanced Green Fluorescence Protein (EGFP) and the mitochondria localization signal can be from glutaredoxin (Grx2). In this context the fluorescence reporter identifies CAR+ T cells with high mitochondrial SRC. For example, the transgenic cells expressing the reporter can be sorted based on intensity fluorescence and infused for tumor killing in vivo. Likewise, the transgenic cells could be tested for ex vivo killing of target cells to determine, for example, the therapeutic effectiveness of a candidate CAR polypeptide.

In some aspects, the mitochondrial reporter gene for use according to the embodiments may be an endogenous gene. In further aspects, the mitochondrial reporter gene may be an exogenous gene, such as a gene encoding a fluorescent reporter protein. In some aspects, the fluorescent reporter protein may comprise a mitochondrial localization sequence. In certain aspects, a method for selecting immune effector cells having high SRC may comprise flow cytometry or FACS.

In certain aspects, expression of the reporter gene for identifying immune effector cells with SRC may be under the control of a nuclear promoter (e.g., hEF1a). In certain aspects, expression of the reporter gene may be under the control of a mitochondrial promoter. In certain aspects, the expressed reporter protein may comprise a mitochondrial localization sequence In certain aspects, the expressed reporter protein may be directed to the cell surface. In certain aspects, expression of the reporter gene may be under the control of a mitochondrial promoter and the expressed reporter protein may be directed to the cell surface. In some aspects, an exogenous reporter gene may be flanked by a transposon repeat or a viral LTR. In some aspects, an exogenous reporter gene may be comprised in an extrachromosomal nucleic acid, such as an mRNA or an episomal vector.

### VII. Methods for Propagating Immune Effector Cells

In some cases, immune effector cells of the embodiments (e.g., T-cells) are co-cultured with activating and propagating cells (AaPCs), to aid in cell expansion. For example, antigen presenting cells (APCs) are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Pat. Application Publication Nos. 2009/0017000 and 2009/0004142; and International Publication No. WO2007/103009, each of which is incorporated by reference.

In some cases, AaPCs express an antigen of interest (e.g., a CoV spike protein). Furthermore, in some cases, APCs can express an antibody that binds to either a specific CAR polypeptide or to CAR polypeptides in general (e.g., a universal activating and propagating cell (uAPC). Such methods are disclosed in International (PCT) Patent Pub. no. WO/2014/190273, which is incorporated herein by reference. In addition to antigens of interest, the AaPC systems may also comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD70 and B7.1 (B7.1 was previously known as B7 and also known as CD80), which among other things, bind to CD28 and/or CTLA-4 molecules on the surface of T cells, thereby affecting, for example, T-cell expansion, Th1 differentiation, short-term T-cell survival, and cytokine secretion such as interleukin (IL)-2 (see Kim et al., 2004). Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), that promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001, incorporated herein by reference.

Cells selected to become AaPCs, preferably have deficiencies in intracellular antigen-processing, intracellular peptide trafficking, and/or intracellular MHC Class I or Class II molecule-peptide loading, or are poikilothermic (i.e., less sensitive to temperature challenge than mammalian cell lines), or possess both deficiencies and poikilothermic properties. Preferably, cells selected to become AaPCs also lack the ability to express at least one endogenous counterpart (e.g., endogenous MHC Class I or Class II molecule and/or endogenous assisting molecules as described above) to the exogenous MHC Class I or Class II molecule and assisting molecule components that are introduced into the cells. Furthermore, AaPCs preferably retain the deficiencies and poikilothermic properties that were possessed by the cells prior to their modification to generate the AaPCs. Exemplary AaPCs either constitute or are derived from a transporter associated with antigen processing (TAP)-deficient cell line, such as an insect cell line. An exemplary poikilothermic insect cells line is a Drosophila cell line, such as a Schneider 2 cell line (see, e.g., Schneider 1972) Illustrative methods for the preparation, growth, and culture of Schneider 2 cells, are provided in U.S. Pat. Nos. 6,225,042, 6,355,479, and 6,362,001.

In one embodiment, AaPCs are also subjected to a freeze-thaw cycle. In an exemplary freeze-thaw cycle, the AaPCs may be frozen by contacting a suitable receptacle containing the AaPCs with an appropriate amount of liquid nitrogen, solid carbon dioxide (i.e., dry ice), or similar low-temperature material, such that freezing occurs rapidly. The frozen APCs are then thawed, either by removal of the AaPCs from the low-temperature material and exposure to ambient room temperature conditions, or by a facilitated thawing process in which a lukewarm water bath or warm hand is employed to facilitate a shorter thawing time. Additionally, AaPCs may be frozen and stored for an extended period of time prior to thawing. Frozen AaPCs may also be thawed and then lyophilized before further use. Preferably, preservatives that might detrimentally impact the freeze-thaw procedures, such as dimethyl sulfoxide (DMSO), polyethylene glycols (PEGs), and other preservatives, are absent from media containing AaPCs that undergo the freeze-thaw cycle, or are essentially removed, such as by transfer of AaPCs to media that is essentially devoid of such preservatives.

In further embodiments, xenogenic nucleic acid and nucleic acid endogenous to the AaPCs, may be inactivated by crosslinking, so that essentially no cell growth, replication or expression of nucleic acid occurs after the inactivation. In one embodiment, AaPCs are inactivated at a point subsequent to the expression of exogenous MHC and assisting molecules, presentation of such molecules on the surface of the AaPCs, and loading of presented MHC molecules with selected peptide or peptides. Accordingly, such inactivated and selected peptide loaded AaPCs, while rendered essentially incapable of proliferating or replicating, retain selected peptide presentation function. Preferably, the crosslinking also yields AaPCs that are essentially free of contaminating microorganisms, such as bacteria and viruses, without substantially decreasing the antigen-presenting cell function of the AaPCs. Thus crosslinking maintains the important AaPC functions of while helping to alleviate concerns about safety of a cell therapy product developed using the AaPCs. For methods related to crosslinking and AaPCs, see for example, U.S. Pat. Application Publication No. 20090017000, which is incorporated herein by reference.

### VIII. Therapeutic Applications

In some aspects, the CAR bridging proteins and chimeric antigen receptor constructs and cells of the embodiments find application in subjects having or suspected of having a coronavirus infection. Suitable immune effector cells that can be used include cytotoxic lymphocytes (CTL). As is well-known to one of skill in the art, various methods are readily available for isolating these cells from a subject. For example, using cell surface marker expression or using commercially available kits (e.g., ISOCELL™ from Pierce, Rockford, Ill.).

Once it is established that the transfected or transduced immune effector cell (e.g., T cell) is capable of expressing the chimeric antigen receptor as a surface membrane protein with the desired regulation and at a desired level, it can be determined whether the chimeric antigen receptor is functional in the host cell to provide for the desired signal induction. Subsequently, the transduced immune effector cells are reintroduced or administered to the subject to activate anti-tumor responses in the subject. To facilitate administration, the transduced T cells according to the embodiments can be made into a pharmaceutical composition or made into an implant appropriate for administration in vivo, with appropriate carriers or diluents, which further can be pharmaceutically acceptable. The means of making such a composition or an implant have been described in the art (see, for instance, Remington’s Pharmaceutical Sciences, 16th Ed., Mack, ed., 1980). Where appropriate, the transduced T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not ineffectuate the cells expressing the chimeric antigen receptor. Thus, desirably the transduced T cells can be made into a pharmaceutical composition containing a balanced salt solution, preferably Hanks’ balanced salt solution, or normal saline.

In certain embodiments, CAR-expressing cells of the embodiments are delivered to an individual in need thereof, such as an individual that has cancer or an infection. The cells then enhance the individual’s immune system to attack the respective cancer or pathogen-infected cells. In some cases, the individual is provided with one or more doses of the antigen-specific CAR cells. In cases where the individual is provided with two or more doses of the antigen-specific CAR cells, the duration between the administrations should be sufficient to allow time for propagation in the individual, and in specific embodiments the duration between doses is 1, 2, 3, 4, 5, 6, 7, or more days. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

In certain embodiments, the CAR-expressing cells are delivered to an individual in need thereof prior to the delivery of a bridging protein. In some cases, the duration between the administration of the CAR-expressing cells and the bridging protein may be 1 day, 2 days, 3 days, 4 days, 5 days, 6 days, 7 days, 8 days, 9 days, 10 days, 2 weeks, 3 weeks, 4 weeks, 5 weeks, 6 weeks, 1 month, 2 months, 3 months, 4 months, 5 months, 6 months, 7 months, 8 months, 9 months, or more. In some cases, the individual is provided with one or more doses of the CAR-expressing cells and/or the bridging protein. In cases where the individual is provided with two or more doses of the CAR-expressing cells and/or the bridging protein, the duration between the administrations between doses may be 1, 2, 3, 4, 5, 6, 7, or more days.

In certain embodiments, the CAR-expressing cells are delivered to an individual in need thereof after the delivery of a bridging protein. In some cases, the duration between the administration of the bridging protein and the CAR-expressing cells may be 1 day, 2 days, 3 days, 4 days, 5 days, 6 days, 7 days, 8 days, 9 days, 10 days, 2 weeks, 3 weeks, 4 weeks, 5 weeks, 6 weeks, 1 month, 2 months, 3 months, 4 months, 5 months, 6 months, 7 months, 8 months, 9 months, or more. In some cases, the individual is provided with one or more doses of the CAR-expressing cells and/or the bridging protein. In cases where the individual is provided with two or more doses of the CAR-expressing cells and/or the bridging protein, the duration between the administrations between doses may be 1, 2, 3, 4, 5, 6, 7, or more days.

In certain embodiments, the CAR-expressing cells are delivered to an individual in need thereof simultaneously with the delivery of a bridging protein. In some cases, the individual is provided with one or more doses of the CAR-expressing cells and/or the bridging protein. The second or more delivery may be of only CAR-expressing cells, only of bridging protein, or of a combination of the two. In cases where the individual is provided with two or more doses of the CAR-expressing cells and/or the bridging protein, the duration between the administrations between doses may be 1, 2, 3, 4, 5, 6, 7, or more days.

In some cases, a patient that has been previously treated with CAR-expressing cells may be treated with a bridging protein to re-direct the effector functions of the CAR-expressing cells. In some cases, a patient that has been previously treated with CAR-expressing cells and a bridging protein may be treating with a different bridging protein to re-direct the effector functions of the CAR-expressing cells. This may be done to treat a new tumor or a new infection in the patient. This may be done in the case of antigen loss.

In any of the provided embodiments, a patient may be treated with more than one bridging protein in order to direct the effector functions of the CAR-expressing cells to multiple targets.

A pharmaceutical composition of the embodiments (e.g., comprising CAR-expressing T-cells) can be used alone or in combination with other well-established agents useful for treating cancer. Whether delivered alone or in combination with other agents, the pharmaceutical composition of the embodiments can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be used for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

A composition of the embodiments can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the embodiments, alone or in combination with other active agents, calculated in an amount sufficient to produce the desired effect, in association with a pharmaceutically acceptable diluent, carrier, or vehicle, where appropriate. The specifications for the unit dosage forms of the embodiments depend on the particular pharmacodynamics associated with the pharmaceutical composition in the particular subject.

Desirably an effective amount or sufficient number of the isolated transduced T cells is present in the composition and introduced into the subject such that long-term, specific, anti-tumor responses are established to reduce the size of a tumor or eliminate tumor growth or regrowth than would otherwise result in the absence of such treatment. Desirably, the amount of transduced T cells reintroduced into the subject causes a 10%, 20%, 30%, 40%, 50%, 60%, 70%, 80%, 90%, 95%, 98%, or 100% decrease in tumor size when compared to otherwise same conditions wherein the transduced T cells are not present. As used herein the term “anti-tumor effective amount” refers to an effective amount of CAR-expressing immune effector cells to reduce cancer cell or tumor growth in a subject.

Accordingly, the amount of transduced immune effector cells (e.g., T cells) administered should take into account the route of administration and should be such that a sufficient number of the transduced immune effector cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1 × 10⁶ to about 1 × 10⁹ transduced T cells, even more desirably, from about 1 × 10⁷ to about 5 × 10⁸ transduced T cells, although any suitable amount can be utilized either above, e.g., greater than 5 × 10⁸ cells, or below, e.g., less than 1 × 10⁷ cells. The dosing schedule can be based on well-established cell-based therapies (see, e.g., Topalian and Rosenberg, 1987; U.S. Pat. No. 4,690,915), or an alternate continuous infusion strategy can be employed.

These values provide general guidance of the range of transduced T cells to be utilized by the practitioner upon optimizing the method of the embodiments. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### IX. Kits of the Embodiments

Any of the compositions described herein may be comprised in a kit. In some embodiments, CAR bridging proteins and/or CAR-expressing immune effector cells are provided in the kit, which also may include reagents suitable for expanding the cells, such as media, APCs, engineered APCs, growth factors, antibodies (e.g., for sorting or characterizing CAR-expressing cells) and/or plasmids encoding transgenes.

In a non-limiting example, a chimeric antigen receptor expression construct, one or more reagents to generate a chimeric antigen receptor expression construct, cells for transfection of the expression construct, and/or one or more instruments to obtain allogeneic cells for transfection of the expression construct (such an instrument may be a syringe, pipette, forceps, and/or any such medically approved apparatus).

In some embodiments, an expression construct for eliminating endogenous TCR α/β expression and/or MHC expression (e.g., beta-2 microglobulin), one or more reagents to generate the construct, and/or CAR+ cells are provided in the kit. In some embodiments, there includes expression constructs that encode zinc finger nuclease(s).

In some aspects, the kit comprises reagents or apparatuses for electroporation of cells.

The kits may comprise one or more suitably aliquoted compositions of the embodiments or reagents to generate compositions of the embodiments. The components of the kits may be packaged either in aqueous media or in lyophilized form. The container means of the kits may include at least one vial, test tube, flask, bottle, syringe, or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits of the embodiments also will typically include a means for containing the chimeric antigen receptor construct and any other reagent containers in close confinement for commercial sale. Such containers may include injection or blow molded plastic containers into which the desired vials are retained, for example.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 - Construction of CAR-Antigen Bridging Protein

The inventors sought to develop a bridge protein to re-direct HIV-specific CAR T cells to an antigen expressed on tumour cells, and thereby demonstrate killing of these target cells. To this end, a bridging protein was created by conjugating or fusing gp 120t to an antigen binding domain, such as an antibody that binds to a target antigen of interest.

### A. Methodology

Construct design and molecular cloning. Anti-HIV CAR T cells were previously developed based on the use of a truncated CD4 (CD4t) extracellular domain (CAR4), which recognizes HIV envelope (env, gp120) protein. Unlike the full-length CD4 glycoprotein, which contains four immunoglobulin domains (D1 to D4), CD4t makes use of a truncated CD4 protein that consists of the D1 and D2 domains. Therefore, the CAR-modified cells will bind HIV env on infected cells. The essential CAR4 design is presented in FIG. 5.

Lentiviral vector production. Lentiviral vectors were produced by transfecting HEK293T cells with CAR-carrying plasmids, as well as lentiviral packaging plasmids PAX2 and VSVg. The cell culture medium was replenished at 4-6 hours and subsequently harvested at 12-24-48 hours for viral particle collection. The culture medium was collected, filtered to remove cellular debris, and viral particles enriched using ultracentrifugation (19,500 rpm, 2 hours). Final aliquots of concentrated lentiviral vectors were stored at -80° C. Functional viral vector titres were assessed by transducing HT1080 cells over a range of dilutions and measuring the percentage of cells expressing the RQR8.

Cells and cell lines. Primary T cells were prepared from anonymised buffy coat blood units procured from the Blood Transfusion Centre of the University Hospital of Geneva. Peripheral blood mononuclear cells (PBMCs) were isolated using Ficoll, T cells separated using Miltenyi CD4/CD8 microbeads and cryopreserved in aliquots in liquid nitrogen. In these proof of principle investigations, HL-60 cells transduced to express CD117 were used as target cells.

CAR T cell manufacturing. Cryopreserved T cells were thawed, cultured overnight in TexMACS medium, and activated the following day using either CD3/CD28 microbeads (1:1 ratio) or TransAct (Miltenyi), and virally transduced with CAR constructs at a multiplicity of infection (MOI) of 3-7. Transductions were performed in high density volumes (2 million cells per ml per cm²), and the medium replenished after 18-24 hours and every other day thereafter for T-cell maintenance at a cell density 1 million per mL.

Flow cytometry was performed 5-7 days post-transduction of T cells. Cells were harvested, washed, resuspended in FACS buffer (Ca/Mg2+ Free PBS, 2 mM EDTA, 0.5% BSA) and stained for 20-30 min with CD34 antibody (QBEnd10) to assess the frequency of reporter gene (RQR8) expressing cells. Following staining, cells were washed with PBS, resuspended in FACS buffer, and cell surface expression was assessed via flow cytometry.

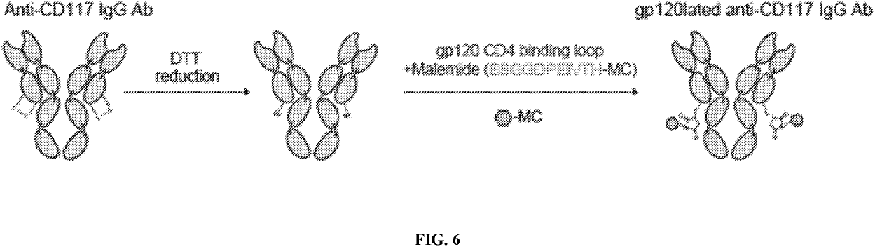

Bridge protein design and production. A bridge protein construct was designed based on the chemical conjugation of a truncated glycoprotein 120 (gp120t) to IgG and diabody antibody formats. A truncated gp120 fragment of 11 amino acids was chemically synthesized (SSGGDPEIVTH; SEQ ID NO: 6) with a maleimide loop. To allow for conjugation, IgG and diabody proteins were produced with cysteine residues. Chemical conjugation was initiated with dithiothreitol (DTT) reduction and the addition gp120t-maleimide (FIG. 6).

Cytotoxicity assays. CAR4 T cells were co-cultured for 18-24 hours with target cells at an effector to target ratio of 1:1. Conjugated antibodies were also added to a final concentration of 500 nM. The ability of CAR4 T cells to bind to the tumor-associated antigen (CD117 in this case) on HL-60 cells was assessed by measuring the proportional decreases in the percentage of viable target cells remaining in the co-cultures.

### B. Results

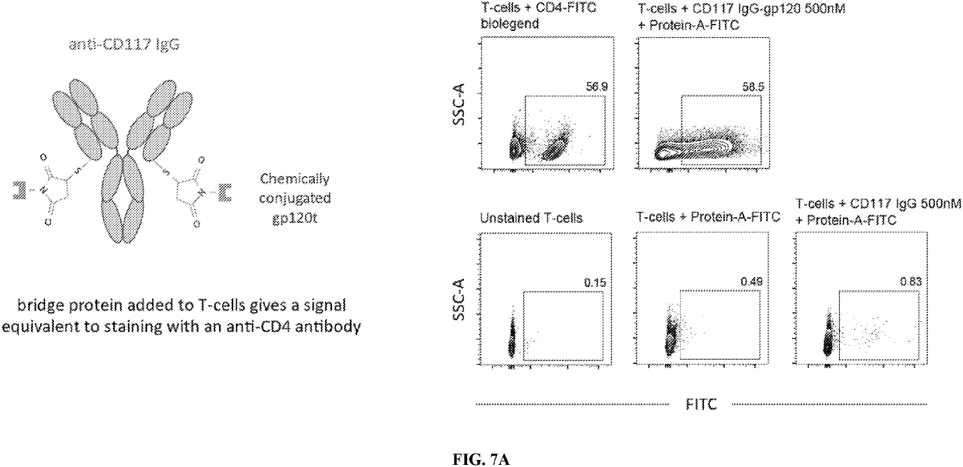

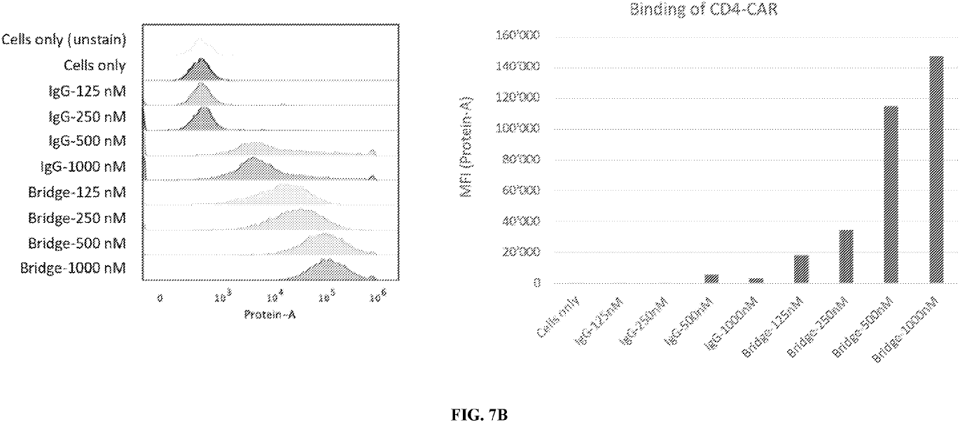

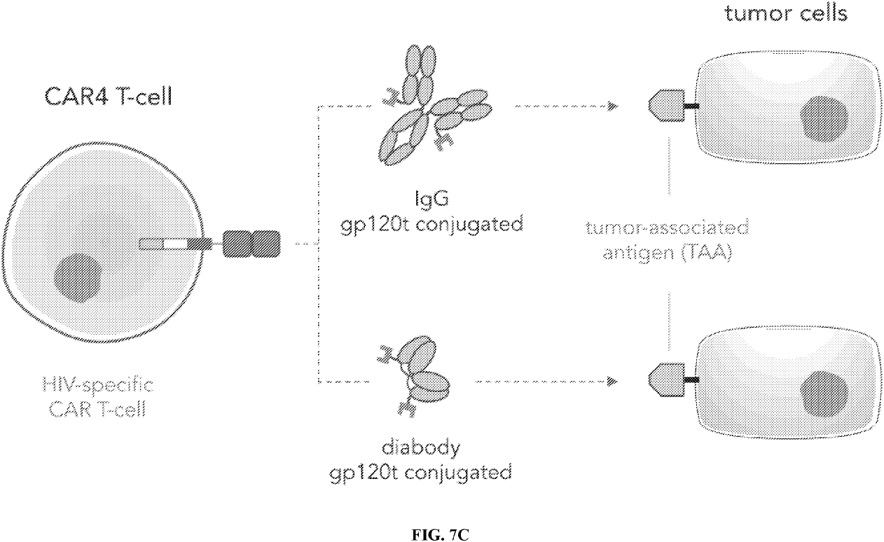

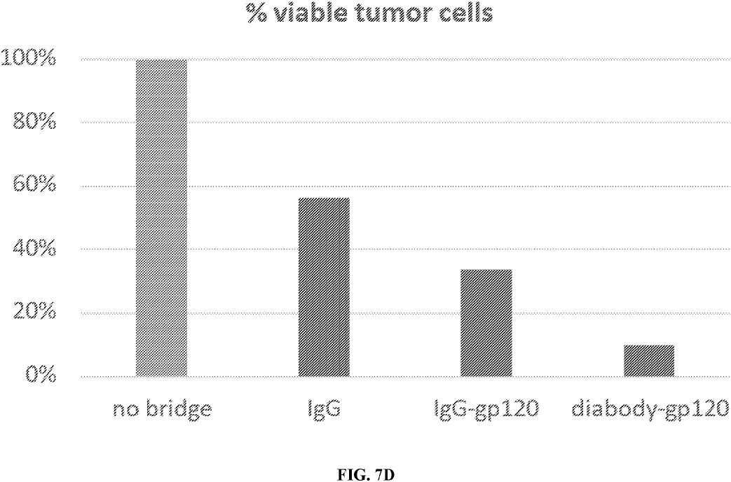

Bridge protein binds CD4 and is redirected to kill tumour cells. The inventors first set out to demonstrate successful conjugation of gp120t to an IgG, and binding to CD4 protein expressed on T cells (FIG. 7A). To test this, primary T cells were exposed to varying concentrations of the IgG-conjugates for 30 min, followed by two washes to remove unbound IgG, and staining with FITC-labelled protein A for detection of CD4-bound IgG protein. The IgG-conjugate proteins were able to bind natural CD4 on primary T cells, and importantly, recapitulate an equivalent percentage of CD4 positive cells when compared to using an anti-CD4 antibody (58.5% and 56.9%, respectively).

Next, a similar experiment was performed to confirm binding of the IgG-conjugate to CAR4 receptors (FIG. 7B). For this, HT1080 cells were transduced with lentiviral vectors carrying a CAR4 construct, and the cells exposed to varying concentrations of either IgG or IgG-conjugated proteins. As seen in flow cytometric histograms in FIG. 7B, there was an evident and proportional increase in the median fluorescent intensity (MFI) of FITC-labelled Protein-A when using the IgG-conjugated bridge proteins, but which was not observed when using IgG alone.

Finally, the inventors sought to demonstrate that the bridge proteins were able to re-direct CAR4 T cells to target and kill tumour cells (FIGS. 7C and 7D). In this experiment, CD117-expressing HL-60 tumour cells were co-cultured with CAR4 T-cells (1:1 ratio) and various bridge protein configurations (500 nM). The inventors also created an anti-CD117 diabody, which was conjugated with gp120t in this assessment. Following a 24 co-culture, significantly increased cytotoxicity of target cells was observed when using IgG-conjugated (65%) and diabody-conjugated (90%) bridge proteins when normalized to controls (CAR4 T cells only, no bridge proteins). This confirmed that the bridge proteins were able to effectively bind CAR4 T-cells and re-direct them toward tumor cells such that they elicit specific cytotoxicity.

### C. Summary and Conclusion

This confirms that an antibody conjugate capable of bridging HIV-specific CAR T cells to tumor cells expressing an antigen of interest serves to re-direct the cytotoxicity of the HIV-specific CAR T cells. Once engaged, the CAR T cells demonstrated effective killing of tumor cells, which was more pronounced with the use of a diabody-gp120t conjugate. Future experiments include re-directing CAR4 T-cells to other relevant tumour-associated antigens, including CD19, CD20 and CD22 for B-cell malignancies, as well as antigens expressed on solid tumours.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. App. Publ. No. 2002/0168707
U.S. Pat. App. Publ. No. 2003/0051263
U.S. Pat. App. Publ. No. 2003/0055020
U.S. Pat. App. Publ. No. 2003/0159161
U.S. Pat. App. Publ. No. 2004/0064842
U.S. Pat. App. Publ. No. 2004/0265839
U.S. Pat. App. Publ. No. 2009/0004142
U.S. Pat. App. Publ. No. 2009/0017000
U.S. Pat. No. 4,690,915
U.S. Pat. No. 5,302,523
U.S. Pat. No. 5,322,783
U.S. Pat. No. 5,384,253
U.S. Pat. No. 5,464,765
U.S. Pat. No. 5,486,359
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,908,782
U.S. Pat. No. 5,945,100
U.S. Pat. No. 5,981,274
U.S. Pat. No. 5,994,136
U.S. Pat. No. 5,994,624
U.S. Pat. No. 6,013,516
U.S. Pat. No. 6,225,042
U.S. Pat. No. 6,355,479
U.S. Pat. No. 6,362,001
U.S. Pat. No. 6,410,319
U.S. Pat. No. 6,506,559
U.S. Pat. No. 6,573,099
U.S. Pat. No. 6,790,662
EP1507865
WO 94/09699
WO 95/06128
WO 96/39487
WO 2005/028630
WO 2007/103009
WO 2010/079430
WO 2014/190273
WO 2015/123642
WO 2019/186274

Angel et al., “12-0-tetradecanoyl-phorbol-13-acetate Induction of the Human Collagenase Gene is Mediated by an Inducible Enhancer Element Located in the 5′ Flanking Region,” Mol. Cell. Biol., 7:2256-2266, 1987a.

Angel et al., “Phorbol Ester-Inducible Genes Contain a Common cis Element Recognized by a TPA-Modulated Trans-acting Factor,” Cell, 49:729-739, 1987b.

Atchison and Perry, “Tandem Kappa Immunoglobulin Promoters are Equally Active in the Presence of the Kappa Enhancer: Implications for Model of Enhancer Function,” Cell, 46:253-262, 1986.

Ausubel et al., Current Protocols in Molecular Biology, Greene Publ. Assoc. Inc. & John Wiley & Sons, Inc., MA, 1996.

Banerji et al., “A lymphocyte-specific cellular enhancer is located downstream of the joining region in immunoglobulin heavy-chain genes,” Cell, 35:729-740, 1983.

Banerji et al., “Expression of a Beta-Globin Gene is Enhanced by Remote SV40 DNA Sequences,” Cell, 27:299-308, 1981.

Berkhout et al., “Tat Trans-activates the Human Immunodeficiency Virus Through a Nascent RNA Target,” Cell, 59:273-282, 1989.

Blanar et al., “A gamma-interferon-induced factor that binds the interferon response sequence of the MHC class I gene, H-2Kb,” EMBO J., 8:1139-1144, 1989.

Blomer et al., J. Virol., 71(9):6641-6649, 1997.

Bodine and Ley, “An enhancer element lies 3′ to the human a γ globin gene,” EMBO J., 6:2997-3004, 1987.

Boshart et al., “A very strong enhancer is located upstream of an immediate early gene of human cytomegalovirus,” Cell, 41:521-530, 1985.

Braddock et al., “HIV-I Tat activates presynthesized RNA in the nucleus,” Cell, 58:269-279, 1989.

Bulla and Siddiqui, “The hepatitis B virus enhancer modulates transcription of the hepatitis B virus surface-antigen gene from an internal location,” J. Virol., 62:1437-1441, 1988.

Campbell and Villarreal, “Functional analysis of the individual enhancer core sequences of polyomavirus: cell-specific uncoupling of DNA replication from transcription,” Mol. Cell. Biol., 8:1993-2004, 1988.

Campo et al., “Transcriptional control signals in the genome of bovine papilloma virus type 1,” Nature, 303:77-80, 1983.

Capecchi, Nature, 348:109, 1990.

Celander and Haseltine, “Glucocorticoid Regulation of Murine Leukemia Virus Transcription Elements is Specified by Determinants Within the Viral Enhancer Region,” J. Virology, 61:269-275, 1987.

Celander et al., “Regulatory Elements Within the Murine Leukemia Virus Enhancer Regions Mediate Glucocorticoid Responsiveness,” J. Virology, 62:1314-1322, 1988.

Chandler et al., “DNA Sequences Bound Specifically by Glucocorticoid Receptor in vitro Render a Heterlogous Promoter Hormone Responsive in vivo,” Cell, 33:489-499, 1983.

Chang et al., “Glucose-regulated Protein (GRP94 and GRP78) Genes Share Common Regulatory Domains and are Coordinately Regulated by Common Trans-acting Factors,” Mol. Cell. Biol., 9:2153-2162, 1989.

Chatterjee et al., “Negative Regulation of the Thyroid-Stimulating Hormone Alpha Gene by Thyroid Hormone: Receptor Interaction Adjacent to the TATA Box,” Proc Natl. Acad Sci. U.S.A., 86:9114-9118, 1989.

Chen and Okayama, “High-efficiency transformation of mammalian cells by plasmid DNA,” Mol. Cell. Biol., 7:2745-2752, 1987.

Chevalier et al., Mol. Cell., 10:895-905, 2002.

Choi et al., “An altered pattern of cross-resistance in multi-drug-resistant human cells results from spontaneous mutations in the mdr-1 (p-glycoprotein) gene,” Cell, 53:519-529, 1989.

Cohen et al., “A Repetitive Sequence Element 3′ of the Human c-Ha-ras 1 Gene Has Enhancer Activity,” J. Cell. Physiol. Suppl., 5:75-81, 1987.

Costa et al., “The Cell-Specific Enhancer of the Mouse Transthyretin (Prealbumin) Gene Binds a Common Factor at One Site and a Liver-Specific Factor(s) at Two Other Sites,” Mol. Cell. Biol., 8:81-90, 1988.

Cripe et al., “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J., 6:3745-3753, 1987.

Culotta and Hamer, “Fine Mapping of a Mouse Metallothionein Gene Metal-Response Element,” Mol. Cell. Biol., 9:1376-1380, 1989.

Dandolo et al., “Regulation of Polyoma Virus Transcription in Murine Embryonal Carcinoma Cells,” J. Virology, 47:55-64, 1983.

De Villiers et al., “Polyoma Virus DNA Replication Requires an Enhancer,” Nature, 312:242-246, 1984.

Deschamps et al., “Identification of a Transcriptional Enhancer Element Upstream From the Proto-Oncogene Fos,” Science, 230:1174-1177, 1985.

Durai et al., Nucleic Acids Res., 33:5978-5990, 2005.

Edbrooke et al., “Identification of cis-acting sequences responsible for phorbol ester induction of human serum amyloid a gene expression via a nuclear-factor-kappa β-like transcription factor,” Mol. Cell. Biol., 9:1908-1916, 1989.

Edlund et al., “Cell-specific expression of the rat insulin gene: evidence for role of two distinct 5′ flanking elements,” Science, 230:912-916, 1985.

Evans, et al., In: Cancer Principles and Practice of Oncology, Devita et al. (Eds.), Lippincot-Raven, N.Y., 1054-1087, 1997.

Fechheimer et al., “Transfection of mammalian cells with plasmid DNA by scrape loading and sonication loading,” Proc Nat′l. Acad. Sci. USA 84:8463-8467, 1987.

Feng and Holland, “HIV-I Tat Trans-Activation Requires the Loop Sequence Within Tar,” Nature, 334(6178): 165-167, 1988.

Firak and Subramanian, “Minimal Transcription Enhancer of Simian Virus 40 is a 74-Base-Pair Sequence that Has Interacting Domains,” Mol. Cell. Biol., 6:3667-3676, 1986.

Foecking and Hofstetter, “Powerful and Versatile Enhancer-Promoter Unit for Mammalian Expression Vectors,” Gene, 45(1): 101-105, 1986.

Fraley et al., Proc. Natl. Acad. Sci. USA, 76:3348-3352, 1979.

Fujita et al., “Interferon-β Gene Regulation: Tandemly Repeated Sequences of a Synthetic 6-bp Oligomer Function as a Virus-Inducible Enhancer,” Cell, 49:357-367, 1987.

Ghosh and Bachhawat, In: Liver Diseases, Targeted Diagnosis and Therapy Using Specific Receptors and Ligands, Wu et al. (Eds.), Marcel Dekker, NY, 87-104, 1991.

Gilles et al., “A tissue-specific transcription enhancer element is located in the major intron of a rearranged immunoglobulin heavy-chain gene,” Cell, 33:717-728, 1983.

Giry-Laterriere et al., Hum Gene Ther, 22: 1255-1267, 2011.

Giry-Laterriere et al., Methods in molecular biology, 737: 183-209, 2011.

Gloss et al., “The Upstream Regulatory Region of the Human Papilloma Virus-16 Contains an E2 Protein-Independent Enhancer Which is Specific for Cervical Carcinoma Cells and Regulated by Glucocorticoid Hormones,” EMBO J., 6:3735-3743, 1987.

Godbout et al., “Fine-Structure Mapping of the Three Mouse Alpha-Fetoprotein Gene Enhancers,” Mol. Cell. Biol., 8:1169-1178, 1988.

Goodbourn and Maniatis, “Overlapping Positive and Negative Regulatory Domains of the Human β-Interferon Gene,” Proc. Natl. Acad. Sci. USA, 85:1447-1451, 1988.

Goodbourn et al., “The Human Beta-Interferon Gene Enhancer is Under Negative Control,” Cell, 45:601-610, 1986.

Gopal, “Gene transfer method for transient gene expression, stable transformation, and cotransformation of suspension cell cultures,” Mol. Cell. Biol. 5:1188-1190, 1985.

Graham and Van Der Eb, “A new technique for the assay of infectivity of human adenovirus 5 DNA,” Virology, 52:456-467, 1973.

Greene et al., “HIV-1, and Normal T-Cell Growth: Transcriptional Strategies and Surprises,” Immunology Today, 10:272-278, 1989.

Grosschedl and Baltimore, “Cell-Type Specificity of Immunoglobulin Gene Expression is Regulated by at Least Three DNA Sequence Elements,” Cell, 41:885-897, 1985.

Harland and Weintraub, J. Cell Biol., 101(3):1094-1099, 1985.

Haslinger and Karin, “Upstream Promoter Element of the Human Metallothionein-II Gene Can Act Like an Enhancer Element,” Proc Natl. Acad. Sci. U.S.A., 82:8572-8576, 1985.

Hauber and Cullen, “Mutational Analysis of the Trans-Activation-Responsive Region of the Human Immunodeficiency Virus Type I Long Terminal Repeat,” J. Virology, 62:673-679, 1988.

Hen et al., “A Mutated Polyoma Virus Enhancer Which is Active in Undifferentiated Embryonal Carcinoma Cells is not Repressed by Adenovirus-2 E1A Products,” Nature, 321:249-251, 1986.

Hensel et al., “PMA-Responsive 5′ Flanking Sequences of the Human TNF Gene,” Lymphokine Res., 8:347-351, 1989.

Herr and Clarke, “The SV40 Enhancer is Composed of Multiple Functional Elements That Can Compensate for One Another,” Cell, 45:461-470, 1986.

Hirochika et al., “Enhancers and Trans-Acting E2 Transcriptional Factors of Papilloma Viruses,” J. Virol., 61:2599-2606, 1987.

Holbrook et al., “cis-Acting Transcriptional Regulatory Sequences in the Gibbon Ape Leukemia Virus (GALV) Long Terminal Repeat,” Virology, 157:211-219, 1987.

Horlick and Benfield, “The upstream muscle-specific enhancer of the rat muscle creatine kinase gene is composed of multiple elements,” Mol. Cell. Biol., 9:2396-2413, 1989.

Huang et al., “Glucocorticoid regulation of the ha-musv p21 gene conferred by sequences from mouse mammary tumor virus,” Cell, 27:245-255, 1981.

Hug et al., “Organization of the Murine Mx Gene and Characterization of its Interferon- and Virus-Inducible Promoter,” Mol. Cell. Biol., 8:3065-3079, 1988.

Hwang et al., “Characterization of the S-Phase-Specific Transcription Regulatory Elements in a DNA-Replication-Independent Testis-Specific H2B (TH2B) Histone Gene,” Mol. Cell. Biol., 10:585-592, 1990.

Imagawa et al., “Transcription Factor AP-2 Mediates Induction by Two Different Signal-Transduction Pathways: Protein Kinase C and cAMP,” Cell, 51:251-260, 1987.

Imbra and Karin, “Phorbol Ester Induces the Transcriptional Stimulatory Activity of the SV40 Enhancer,” Nature, 323:555-558, 1986.

Imler et al., “Negative Regulation Contributes to Tissue Specificity of the Immunoglobulin Heavy-Chain Enhancer,” Mol. Cell. Biol, 7:2558-2567, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875-882, 1984.

Jakobovits et al., “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555-2561, 1988.

Jameel and Siddiqui, “The Human Hepatitis B Virus Enhancer Requires Transacting Cellular Factor(s) for Activity,” Mol. Cell. Biol., 6:710-715, 1986.

Jaynes et al., “The Muscle Creatine Kinase Gene is Regulated by Multiple Upstream Elements, Including a Muscle-Specific Enhancer,” Mol. Cell. Biol., 8:62-70, 1988.

Johnson et al., “Muscle creatine kinase sequence elements regulating skeletal and cardiac muscle expression in transgenic mice,” Mol. Cell. Biol., 9:3393-3399, 1989.

Kadesch and Berg, “Effects of the Position of the Simian Virus 40 Enhancer on Expression of Multiple Transcription Units in a Single Plasmid,” Mol. Cell. Biol., 6:2593-2601, 1986.

Kaeppler et al., Plant Cell Rep., 8:415-418, 1990.

Kaneda et al., Science, 243:375-378, 1989.

Karin et al., “Metal-Responsive Elements Act as Positive Modulators of Human Metallothionein-IIA Enhancer Activity,” Mol. Cell. Biol., 7:606-613, 1987.

Karin et al. Cell, 36: 371-379, 1989.

Katinka et al., “Expression of Polyoma Early Functions in Mouse Embryonal Carcinoma Cells Depends on Sequence Rearrangements in the Beginning of the Late Region,” Cell, 20:393-399, 1980.

Kato et al, J. Biol. Chem., 266:3361-3364, 1991.

Kawamoto et al., “Identification of the Human Beta-Actin Enhancer and its Binding Factor,” Mol. Cell. Biol., 8:267-272, 1988.

Kiledjian et al., “Identification and characterization of two functional domains within the murine heavy-chain enhancer,” Mol. Cell. Biol., 8:145-152, 1988.

Kim et al., Gene, 91(2):217-23, 1990.

Kim et al., Nat. Biotechnol ., 22:403-10, 2004.

Klamut et al., “Molecular and Functional Analysis of the Muscle-Specific Promoter Region of the Duchenne Muscular Dystrophy Gene,” Mol. Cell. Biol., 10:193-205, 1990.

Koch et al., “Anatomy of a new B-cell-specific enhancer,” Mol. Cell. Biol., 9:303-311, 1989.

Kriegler and Botchan, “A retrovirus LTR contains a new type of eukaryotic regulatory element,” In: Eukaryotic Viral Vectors, Gluzman (ed.), Cold Spring Harbor, Cold Spring Harbor Laboratory, NY, 171-180, 1982.

Kriegler et al., “A Novel Form of TNF/Cachectin Is a Cell-Surface Cytotoxix Transmembrane Protein: Ramifications for the Complex Physiology of TNF,” Cell, 53:45-53, 1988.

Kriegler et al., “Promoter substitution and enhancer augmentation increases the penetrance of the sv40 a gene to levels comparable to that of the harvey murine sarcoma virus ras gene in morphologic transformation,” In: Gene Expression, Alan Liss (Ed.), Hamer and Rosenberg, New York, 107-124, 1983.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. (eds), Cold Spring Harbor, Cold Spring Harbor Laboratory, 345-353, 1984.

Kuhl et al., “Reversible Silencing of Enhancers by Sequences Derived From the Human IFN-alpha Promoter,” Cell, 50:1057-1069, 1987.

Kunz et al., “Identification of the Promoter Sequences Involved in the Interleukin-6-Dependent Expression of the Rat Alpha-2-Macroglobulin Gene,” Nucl. Acids Res., 17:1121-1138, 1989.

Langle-Rouault et al., J. Virol., 72(7):6181-6185, 1998.

Larsen et al., “Repression mediates cell-type-specific expression of the rat growth hormone gene,” Proc Natl. Acad. Sci. USA., 83:8283-8287, 1986.

Laspia et al., “HIV-1 Tat protein increases transcriptional initiation and stabilizes elongation,” Cell, 59:283-292, 1989.

Latimer et al., “Highly conserved upstream regions of the alpha..sub.1-antitrypsin gene in two mouse species govern liver-specific expression by different mechanisms,” Mol. Cell. Biol., 10:760-769, 1990.

Lee et al., “Glucocorticoids Regulate Expression of Dihydrofolate Reductase cDNA in Mouse Mammary Tumor Virus Chimaeric Plasmids,” Nature, 294:228-232, 1981.

Levinson et al., “Activation of SV40 Genome by 72-Base-Pair Tandem Repeats of Moloney Sarcoma Virus,” Nature, 295:568-572, 1982.

Levitskaya et al., Proc. Natl. Acad. Sci. USA, 94(23):12616-12621, 1997.

Lin et al., “Delineation of an enhancerlike positive regulatory element in the interleukin-2 receptor alpha.-chain gene,” Mol. Cell. Biol., 10:850-853, 1990.

Luria et al., “Promoter Enhancer Elements in the Rearranged Alpha-Chain Gene of the Human T-Cell Receptor,” EMBO J., 6:3307-3312, 1987.

Lusky and Botchan, “Transient Replication of Bovine Papilloma Virus Type 1 Plasmids: cis and trans Requirements,” Proc Natl. Acad. Sci. U.S.A., 83:3609-3613, 1986.

Lusky et al., “Bovine Papilloma Virus Contains an Activator of Gene Expression at the Distal End of the Early Transcription Unit,” Mol. Cell. Biol. 3:1108-1122, 1983.

Majors and Varmus, “A Small Region of the Mouse Mammary Tumor Virus Long Terminal Repeat Confers Glucocorticoid Hormone Regulation on a Linked Heterologous Gene,” Proc. Natl. Acad. Sci. U.S.A., 80:5866-5870, 1983.

Maniatis, et al., Molecular Cloning, A Laboratory Manual, Cold Spring Harbor Press, Cold Spring Harbor, N.Y., 1988.

Mann and Frankel, EMBO J., 10: 1733-1739, 1991. Mann et al., Cell, 33:153-159, 1983.

McNeall et al., “Hyperinducible Gene Expression From a Metallotionein Promoter Containing Additional Metal-Responsive Elements,” Gene, 76:81-88, 1989.

Miksicek et al., “Glucocorticoid Responsiveness of the Transcriptional Enhancer of Moloney Murine Sarcoma Virus,” Cell, 46:283-290, 1986.

Miller et al., Am. J. Clin. Oncol., 15(3):216-221, 1992.

Miller et al., Nat. Biotechnol., 29:143-148, 2011.

Mordacq and Linzer, “Co-localization of Elements Required for Phorbol Ester Stimulation and GLucocorticoid Repression of Proliferin Gene Expression,” Genes and Dev., 3:760-769, 1989.

Moreau et al., “The SV40 base-repair repeat has a striking effect on gene expression both in sv40 and other chimeric recombinants,” Nucl. Acids Res., 9:6047-6068, 1981.

Muesing et al., “Regulation of mRNA accumulation by a human immunodeficiency virus trans-activator protein,” Cell, 48:691-701, 1987.

Nabel et al., Science, 244(4910):1342-1344, 1989.

Naldini et al., “Efficient transfer, integration, and sustained long-term expression of the transgene in adult rat brains injected with a lentiviral vector,” Proc. Natl. Acad. Sci. USA, 93:11382-11388, 1996.

Naldini et al., “In vivo gene delivery and stable transduction of nondividing cells by a lentiviral vector,” Science, 272:263-267, 1996.

Naldini, “Lentiviruses as gene transfer agents for delivery to non-dividing cells,” Current Opinion in Biotechnology, 9:457-463, 1998.

Ng et al., “Regulation of the Human Beta-Actin Promoter by Upstream and Intron Domains,” Nuc. Acids Res., 17:601-615, 1989.

Nicolas and Rubenstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Ondek et al., “Discrete Elements Within the SV40 Enhancer Region Display Different Cell-Specific Enhancer Activities,” EMBO J., 6:1017-1025, 1987.

Omitz et al., “Promoter and enhancer elements from the rat elastase i gene function independently of each other and of heterologous enhancers,” Mol. Cell. Biol. 7:3466-3472, 1987.

Palmiter et al., “Differential regulation of metallothionein-thymidine kinase fusion genes in transgenic mice and their offspring,” Cell, 29:701-710, 1982.

Paskind et al., Virology, 67:242-248, 1975.

Pech et al., “Functional identification of regulatory elements within the promoter region of platelet-derived growth factor 2,” Mol. Cell. Biol., 9(2):396-405, 1989.

Perez-Stable and Constantini, “Roles of fetal Gy-globin promoter elements and the adult β-globin 3′ enhancer in the stage-specific expression of globin genes,” Mol. Cell. Biol., 10:1116-1125, 1990.

Picard and Schaffner, “A Lymphocyte-Specific Enhancer in the Mouse Immunoglobulin Kappa Gene,” Nature, 307:80-82, 1984.

Pingoud and Silva, Nat. Biotechnol., 25:743-744, 2007.

Pinkert et al., “An albumin enhancer located 10 kb upstream functions along with its promoter to direct efficient, liver-specific expression in transgenic mice,” Genes and Dev., 1:268-276, 1987.

Ponta et al., “Hormonal Response Region in the Mouse Mammary Tumor Virus Long Terminal Repeat Can Be Dissociated From the Proviral Promoter and Has Enhancer Properties,” Proc. Natl. Acad. Sci. U.S.A., 82:1020-1024, 1985.

Porton et al., “Immunoglobulin heavy-chain enhancer is required to maintain transfected.gamma.2a gene expression in a pre-b-cell line,” Mol. Cell. Biol., 10:1076-1083, 1990.

Potrykus et al., Mol. Gen. Genet., 199(2):169-177, 1985.

Potter et al., “Enhancer-dependent expression of human k immunoglobulin genes introduced into mouse pre-B lymphocytes by electroporation,” Proc Nat′l Acad. Sci. USA, 81:7161-7165, 1984.

Queen and Baltimore, “Immunoglobulin Gene Transcription is Activated by Downstream Sequence Elements,” Cell, 35:741-748, 1983.

Quinn et al., “Multiple components are required for sequence recognition of the ap1 site in the gibbon ape leukemia virus enhancer,” Mol. Cell. Biol., 9:4713-4721, 1989.

Redondo et al., “A T-Cell-Specific Transcriptional Enhancer Within the Human T-Cell Receptor .delta. Locus,” Science, 247:1225-1229, 1990.

Reisman and Rotter, “Induced Expression From the Moloney Murine Leukemia Virus Long Terminal Repeat During Differentiation of Human Myeloid Cells is Mediated Through its Transcriptional Enhancer,” Mol. Cell. Biol., 9:3571-3575, 1989.

Remington’s Pharmaceutical Sciences, 16th Ed., Mack, ed., 1980.

Resendez Jr., et al., “Identification of highly conserved regulatory domains and protein-binding sites in the promoters of the rat and human genes encoding the stress-inducible 78-kilodalton glucose-regulated protein,” Mol. Cell. Biol., 8:4579-4584, 1988.

Rippe et al., “DNA-mediated gene transfer into adult rat hepatocytes in primary culture,” Mol. Cell Biol., 10:689-695, 1990.

Rittling et al., “AP-1/jun-binding Sites Mediate Serum Inducibility of the Human Vimentin Promoter,” Nuc. Acids Res., 17:1619-1633, 1989.

Rosen et al., “The location of cis-acting regulatory sequences in the human t-cell lymphotropic virus type III (HTLV-111/LAV) long terminal repeat,” Cell, 41:813-823, 1985.

Sakai et al., “Hormone-Mediated Repression: A Negative Glucocorticoid-Response Element From the Bovine Prolactin Gene,” Genes and Dev., 2:1144-1154, 1988.

Sambrook and Russell, Molecular Cloning: A Laboratory Manual, 3rd Ed. Cold Spring Harbor Lab. Press, 2001.

Satake et al., “Biological activities of oligonucleotides spanning the f9 point mutation within the enhancer region of polyoma virus DNA,” J. Virology, 62:970-977, 1988.

Schaffner et al., “Redundancy of Information in Enhancers as a Principle of Mammalian Transcription Control,” J. Mol. Biol., 201:81-90, 1988.

Schneider, J. Embryol. Morph. 27: 353-365, 1972

Searle et al., “Building a metal-responsive promoter with synthetic regulatory elements,” Mol. Cell. Biol., 5:1480-1489, 1985.

Sharp and Marciniak, “HIV Tar: an RNA Enhancer?” Cell, 59:229-230, 1989.

Shaul and Ben-Levy, “Multiple Nuclear Proteins in Liver Cells are Bound to Hepatitis B Virus Enhancer Element and its Upstream Sequences,” EMBO J., 6:1913-1920, 1987.

Sherman et al., “Class II Box Consensus Sequences in the HLA-DR.alpha. Gene: Transcriptional Function and Interaction with Nuclear Proteins,” Mol. Cell. Biol., 9:50-56, 1989.

Silva et al., Meganucleases and other tools for targeted genome engineering, Curr Gene Ther 11(1):11-27, 2011.

Sleigh and Lockett, “SV40 Enhancer Activation During Retinoic-Acid-Induced Differentiation of F9 Embryonal Carcinoma Cells,” J. EMBO, 4:3831-3837, 1985.

Spalholz et al.,“Transactivation of a Bovine Papilloma Virus Transcriptional Regulatory Element by the E2 Gene Product,” Cell, 42:183-191, 1985.

Spandau and Lee, “Trans-Activation of Viral Enhancers by the Hepatitis B Virus X Protein,” J. Virology, 62:427-434, 1988.

Spandidos and Wilkie, “Host-Specificities of Papilloma Virus, Moloney Murine Sarcoma Virus and Simian Virus 40 Enhancer Sequences,” EMBO J., 2:1193-1199, 1983.

Stegmeier F. et al., Proc Natl Acad Sci USA, 102(37):13212-13217, 2005.

Stephens and Hentschel, “The Bovine Papilloma Virus Genome and its Uses as a Eukaryotic Vector,” Biochem. J., 248:1-11, 1987.

Stuart et al., “Identification of Multiple Metal Regulatory Elements in Mouse Metallothionein-I Promoter by Assaying Synthetic Sequences,” Nature, 317:828-831, 1985.

Sullivan and Peterlin, “Transcriptional Enhancers in the HLA-DQ Subregion,” Mol. Cell. Biol., 7:3315-3319, 1987.

Swartzendruber and Lehman, “Neoplastic Differentiation: Interaction of Simian Virus 40 and Polyoma Virus with Murine Teratocarcinoma Cells,” J. Cell. Physiology, 85:179-188, 1975.

Takebe et al., “SRα Promoter: An Efficient and Versatile Mammalian cDNA Expression System Composed of the Simian Virus 40 Early Promoter and the R-U5 Segment of Human T-Cell Leukemia Virus Type 1 Long Terminal Repeat,” Mol. Cell. Biol., 8:466-472, 1988.

Tavernier et al., “Deletion Mapping of the Inducible Promoter of Human IFN-beta Gene,” Nature, 301:634-636, 1983.

Taylor and Kingston, “E1A Trans-Activation of Human HSP70 Gene Promoter Substitution Mutants is Independent of the Composition of Upstream and TATA Elements,” Mol. Cell. Biol., 10: 176-183, 1990.

Taylor and Kingston, “Factor Substitution in a Human HSP70 Gene Promoter: TATA-Dependent and TATA-Independent Interactions,” Mol. Cell. Biol., 10:165-175, 1990.

Taylor et al., “Stimulation of the Human Heat-Shock Protein 70 Promoter in vitro by Simian Virus 40 Large T Antigen,” J. Biol. Chem., 264:16160-16164, 1989.

Temin, In: Gene Transfer, Kucherlapati (Ed.), NY, Plenum Press, 149-188, 1986.

Thiesen et al., “A DNA Element Responsible for the Different Tissue Specificities of Friend and Moloney Retroviral Enhancers,” J. Virology, 62:614-618, 1988.

Topalian and Rosenberg, Acta Haematol., 78 Suppl 1:75-76, 1987.

Tronche et al., “Anatomy of the Rat Albumin Promoter,” Mol. Biol. Med., 7:173-185, 1990.

Tronche et al., “The Rat Albumin Promoter: Cooperation with Upstream Elements is Required When Binding of APF/HNF 1 to the Proximal Element is Partially Impaired by Mutation or Bacterial Methylation,” Mol. Cell. Biol., 9:4759-4766, 1989.

Trudel and Constantini, “A 3′ Enhancer Contributes to the Stage-Specific Expression of the human Beta-Globin Gene,” Genes and Dev., 6:954-961, 1987.

Tur-Kaspa et al., “Use of electroporation to introduce biologically active foreign genes into primary rat hepatocytes,” Mol. Cell Biol., 6:716-718, 1986.

Vannice and Levinson, “Properties of the Human Hepatitis B Virus Enhancer: Position Effects and Cell-Type Nonspecificity,” J. Virology, 62:1305-1313, 1988.

Vasseur et al., “Isolation and Characterization of Polyoma Virus Mutants Able to Develop in Multipotential Murine Embryonal Carcinoma Cells,” Proc Natl. Acad. Sci. U.S.A., 77:1068-1072, 1980.

Wang and Calame, “SV40 enhancer-binding factors are required at the establishment but not the maintenance step of enhancer-dependent transciptional activation,” Cell, 47:241-247, 1986.

Weinberger et al., “Localization of a Repressive Sequence Contributing to B-cell Specificity in the Immunoglobulin Heavy-Chain Enhancer,” Mol. Cell. Biol., 8:988-992, 1988.

Wilson et al., Science, 244: 1344-1346, 1989.

Winoto and Baltimore, “αβ-lineage-specific Expression of the α T-Cell Receptor Gene by Nearby Silencers,” Cell, 59:649-655, 1989. Wong et al., Gene, 10:87-94, 1980.

Wu and Wu, Biochemistry, 27: 887-892, 1988.

Wu and Wu, J. Biol. Chem., 262:4429-4432, 1987.

Yutzey et al., “An Internal Regulatory Element Controls Troponin I Gene Expression,” Mol. Cell. Biol., 9:1397-1405, 1989.

Zufferey et al., “Multiply attenuated lentiviral vector achieves efficient gene delivery in vivo,” Nat. Biotechnol., 15:871-875, 1997.

What is claimed is:

1. A chimeric antigen receptor (CAR) bridging protein comprising (1) an antigen-binding domain and (2) a CAR-binding domain, that comprises at least a portion of an HIV-1 gp 120 protein.

2. The CAR bridging protein of claim 1, wherein the CAR-binding domain is chemically conjugated to the antigen-binding domain.

3. The CAR bridging protein of claim 1, wherein the antigen-binding domain is chemically conjugated to the CAR-binding domain.

4. The CAR bridging protein of claim 1, wherein the antigen-binding domain and the CAR-binding domain are comprised in a fusion protein.

5. The CAR bridging protein of claim 4, further comprising an antibody Fc domain.

6. The CAR bridging protein of claim 5, wherein the Fc domain is positioned between the CAR-binding domain and the antigen-binding domain.

7. The CAR bridging protein of claim 5, wherein the CAR-binding domain is positioned between the antigen-binding domain and the Fc domain.

8. The CAR bridging protein of claim 5, wherein the Fc domain comprises a human Fc domain sequence.

9. The CAR bridging protein of claim 8, wherein the Fc domain comprises a human heavy chain Fc domain sequence.

10. The CAR bridging protein of claim 8, wherein the Fc domain comprises CH2 and CH3 regions of a human heavy chain Fc domain sequence.

11. The CAR bridging protein of claim 8, wherein the Fc domain comprises substitutions relative to the wild-type human heavy chain Fc domain sequence which prevent binding to FcgR receptors.

12. The CAR bridging protein of claim 8, wherein the Fc domain comprises a sequence that is at least 85%, at least 90%, at least 95%, or 100% identical to the sequence provided by SEQ ID NO: 4.

13. The CAR bridging protein of claim 1, further comprising a linker sequence between the antigen binding domain and the CAR-binding domain.

14. The CAR bridging protein of claim 1, wherein the CAR-binding domain comprises the sequence provided in SEQ ID NO: 6.

15. The CAR bridging protein of any one of claims 1-14, wherein the antigen-binding domain binds to a tumor antigen or a viral antigen.

16. The CAR bridging protein of any one of claims 1-15, wherein the antigen-binding domain comprises a peptide that interacts with an antigen of interest.

17. The CAR bridging protein of any one of claims 1-16, wherein the antigen-binding domain comprises an antigen-binding portion of an antibody that recognizes the antigen of interest.

18. The CAR bridging protein of any one of claims 1-17, wherein the antigen-binding domain comprises at least a portion of a ligand that interacts with the antigen of interest.

19. The CAR bridging protein of any one of claims 1-18, wherein the antigen-binding domain is capable of binding to CD19, CD20, or CD22.

20. The CAR bridging protein of any one of claims 1-18, wherein the antigen-binding domain is capable of binding to a coronavirus spike protein.

21. The CAR bridging protein of claim 20, wherein the coronavirus spike protein is a SARS-CoV-1 or SARS-CoV-2 spike protein.

22. The CAR bridging protein of any one of claims 1-21, wherein the antigen-binding domain comprises at least a portion of an ACE2 extracellular domain.

23. The CAR bridging protein of claim 22, wherein the portion of an ACE2 extracellular domain is the ACE2t domain.

24. The CAR bridging protein of claim 23, wherein the ACE2t domain comprises a sequence that is at least 85%, at least 90%, at least 95%, or 100% identical to the sequence of SEQ ID NO: 2.

25. The CAR bridging protein of any one of claims 1-24, further comprising at least one linker sequence between the CAR-binding domain, Fc domain, and/or antigen-binding domain.

26. The CAR bridging protein of claim 25, wherein the CAR bridging protein comprises a linker sequence between each of the CAR-binding domain, Fc domain, and/or antigen-binding domains.

27. The CAR bridging protein of claim 25 or 26, wherein the linker sequence comprises the sequence of GGGS.

28. The CAR bridging protein of any one of claims 25-27, wherein the linker sequence comprises a sequence provided by SEQ ID NO: 6.

29. The CAR bridging protein of any one of claims 1-28, wherein the CAR bridging protein forms a homodimer.

30. A chimeric antigen receptor (CAR) bridging protein comprising a CAR-binding domain and an antigen-binding domain.

31. The CAR bridging protein of claim 30, wherein the CAR-binding domain is chemically conjugated to the antigen-binding domain.

32. The CAR bridging protein of claim 30, wherein the antigen-binding domain is chemically conjugated to the CAR-binding domain.

33. The CAR bridging protein of claim 1, wherein the antigen-binding domain and the CAR-binding domain are comprised in a fusion protein.

34. The CAR bridging protein of claim 30, further comprising an antibody Fc domain.

35. The CAR bridging protein of claim 34, wherein the Fc domain is positioned between the CAR-binding domain and the antigen-binding domain.

36. The CAR bridging protein of claim 34, wherein the CAR-binding domain is positioned between the antigen-binding domain and the Fc domain.

37. The CAR bridging protein of any one of claims 30-36, wherein the CAR-binding domain comprises a peptide that interacts with the extracellular portion of a CAR.

38. The CAR bridging protein of any one of claims 30-37, wherein the CAR-binding domain comprises the antigen-binding portion of an antibody that recognizes the extracellular portion of a CAR.

39. The CAR bridging protein of any one of claims 30-37, wherein the CAR-binding domain comprises at least a portion of a ligand that interacts with the extracellular portion of a CAR.

40. The CAR bridging protein of any one of claims 30-37, wherein the CAR-binding domain binds to a portion of the CAR that is specific for the target of the CAR.

41. The CAR bridging protein of claim 40, wherein the CAR comprises scFv and wherein the CAR-binding domain binds to a variable region of the scFv.

42. The CAR bridging protein of any one of claims 30-37, wherein the CAR-binding domain comprises an antibody or an antigen binding fragment thereof.

43. The CAR bridging protein of claim 42, wherein the CAR-binding domain comprises scFv.

44. The CAR bridging protein of any one of claims 30-37, wherein the CAR-binding domain comprises at least a portion of an HIV-1 gp120 protein.

45. The CAR bridging protein of claim 44, wherein the CAR-binding domain comprises the sequence provided in SEQ ID NO: 6.

46. The CAR bridging protein of any one of claims 30-37, wherein the CAR is a CD19 specific CAR and the CAR binding domain binds to the CD19-specific CAR.

47. The CAR bridging protein of claim 46, wherein the CAR binding domain comprises an antibody or an antigen binding fragment thereof.

48. The CAR bridging protein of claim 47, wherein the CAR binding domain comprises a scFv.

49. The CAR bridging protein of claim 46, wherein the CAR-binding domain comprises at least a portion of a CD19 protein.

50. The CAR bridging protein of any one of claims 34-49, wherein the Fc domain comprises a human Fc domain sequence.

51. The CAR bridging protein of any one of claims 34-50, wherein the Fc domain comprises a human heavy chain Fc domain sequence.

52. The CAR bridging protein of any one of claims 34-51, wherein the Fc domain comprises CH2 and CH3 regions of a human heavy chain Fc domain sequence.

53. The CAR bridging protein of any one of claims 34-52, wherein the Fc domain comprises substitutions relative to the wild-type human heavy chain Fc domain sequence which prevent binding to FcgR receptors.

54. The CAR bridging protein of any one of claims 34-53, wherein the Fc domain comprises a sequence that is at least 85%, at least 90%, at least 95%, or 100% identical to the sequence provided by SEQ ID NO: 4.

55. The CAR bridging protein of claim 30, wherein the antigen-binding domain binds to a tumor antigen or a viral antigen.

56. The CAR bridging protein of any one of claims 30-55, wherein the antigen-binding domain comprises a peptide that interacts with an antigen of interest.

57. The CAR bridging protein of any one of claims 30-56, wherein the antigen-binding domain comprises an antigen-binding portion of an antibody that recognizes the antigen of interest.

58. The CAR bridging protein of any one of claims 30-57, wherein the antigen-binding domain comprises at least a portion of a ligand that interacts with the antigen of interest.

59. The CAR bridging protein of any one of claims 30-58, wherein the antigen-binding domain binds to CD19, CD20, or CD22.

60. The CAR bridging protein of any one of claims 30-58, wherein the antigen-binding domain is capable of binding to a coronavirus spike protein.

61. The CAR bridging protein of claim 60, wherein the coronavirus spike protein is a SARS-CoV-1 or SARS-CoV-2 spike protein.

62. The CAR bridging protein of any one of claims 30-61, wherein the antigen-binding domain comprises at least a portion of an ACE2 extracellular domain.

63. The CAR bridging protein of claim 62, wherein the portion of an ACE2 extracellular domain is the ACE2t domain.

64. The CAR bridging protein of claim 63, wherein the ACE2t domain comprises a sequence that is at least 85%, at least 90%, at least 95%, or 100% identical to the sequence of SEQ ID NO: 2.

65. The CAR bridging protein of any one of claims 34-64, further comprising at least one linker sequence between the CAR-binding domain, Fc domain, and/or antigen-binding domain.

66. The CAR bridging protein of any one of claims 34-65, wherein the CAR bridging protein comprises a linker sequence between the CAR-binding domain and the antigen-binding domain, and optionally, the Fc domain.

67. The CAR bridging protein of claim 65 or 66, wherein the linker sequence comprises the sequence of GGGS.

68. The CAR bridging protein of any one of claims 65-67, wherein the linker sequence comprises a sequence provided by SEQ ID NO: 6.

69. The CAR bridging protein of any one of claims 30-68, wherein the CAR bridging protein forms a homodimer.

70. A nucleic acid molecule encoding a CAR bridging protein in accordance with any one of claims 1-69.

71. The nucleic acid molecule of claim 70, wherein the sequence encoding the CAR bridging protein is operatively linked to an expression control sequence.

72. The nucleic acid molecule of claim 70, further defined as an expression vector.

73. The nucleic acid molecule of claim 72, wherein the expression vector is an episomal vector.

74. The nucleic acid molecule of claim 72, wherein the expression vector is a viral vector.

75. The nucleic acid molecule of claim 74, wherein the viral vector is an adenovirus, adeno-associated virus, retrovirus or lentivirus vector.

76. A pharmaceutical composition comprising a CAR bridging protein in accordance with any one of claims 1-69 in a pharmaceutically acceptable carrier.

77. The pharmaceutical composition of claim 76, further comprising a population of immune effector cells comprising a CAR polypeptide that the CAR-binding domain of the CAR bridging protein binds.

78. A method of treating a subject in need thereof, the method comprising administering to the subject an effective amount of a CAR bridging protein in accordance with any one of claims 1-69.

79. The method of claim 78, wherein the subject has previously been administered a population of immune effector cells comprising a CAR polypeptide that the CAR-binding domain of the CAR bridging protein binds.

80. The method of claim 78, further comprising administering to the subject an effective amount of a population of immune effector cells comprising a CAR polypeptide that the CAR-binding domain of the CAR bridging protein binds.

81. The method of claim 80, wherein the cells are allogeneic to the subject.

82. The method of claim 80, wherein the cells are autologous to the subject.

83. The method of claim 80, wherein the cells are HLA matched to the subject.

84. The method of any one of claims 78-83, wherein the subject has a coronavirus infection.

85. The method of any one of claims 78-84, wherein the subject has a SAR-CoV infection.

86. The method of any one of claims 78-84, wherein the subject has a SAR-CoV-2 infection.

87. The method of any one of claims 78-84, wherein the subject has COVID-19.

88. The method of claim 86 or 87, wherein the CAR bridging protein comprises (i) an antigen-binding domain that is at least 85%, at least 90%, at least 95%, or 100% identical to the sequence of SEQ ID NO: 2; and (ii) a CAR-binding domain that is comprises the sequence provided in SEQ ID NO: 6, and wherein the CAR polypeptide comprises a CD4 domain as its antigen-binding domain.

89. The method of any one of claims 78-83, wherein the subject has a cancer.

90. The method of claim 89, wherein the CAR bridging protein comprises an antigen-binding domain that is capable of binding to CD19, CD20, or CD22.

91. The method of claim 78, wherein the CAR-binding domain of the CAR bridging protein comprises at least a portion of a CD19 protein.
